(19) United States
(12) Patent Application Publication
(10) Pub. No.: US 2009/0005436 A1
(43) Pub. Date: Jan. 1, 2009

(54) Substituted Aminoalkyl- and Amidoalkyl-Benzopyran Derivatives

(75) Inventors: Angelo Carotti, Bari (IT); Piero Melloni, Bresso (IT); Florian Thaler, Merano (IT); Carla Caccia, Cardano Al Campo (IT); Sara Maestroni, Turate (IT); Patricia Salvati, Arese (IT)

Correspondence Address: OBLON, SPIVAK, MCCLELLAND MAIER & NEUSTADT, P.C., 1940 DUKE STREET, ALEXANDRIA, VA, 22314, US

(73) Assignee: NEWRON PHARMACEUTICALS S.p.A., Bresso (MI) (IT)

(21) Appl. No.: 11/909,095

(22) PCT Filed: Feb. 22, 2006
(86) PCT No.: PCT/EP2006/001572

§ 371 Date: Sep. 19, 2007

(30) Foreign Application Priority Data
Mar. 29, 2005 (EP) 05006752.9

(51) Int. Cl. A61K 31/353; C07D 311/04; A61P 25/28

(52) U.S. Cl. 514/457; 549/407; 549/401; 514/456

(57) ABSTRACT

This invention is related to novel aminoalkyl- and amidoalkyl-benzopyran derivatives of the following general formula (I)

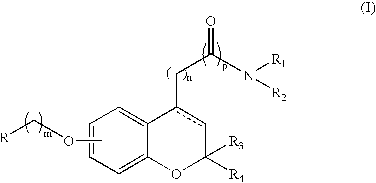

wherein:
the group

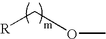

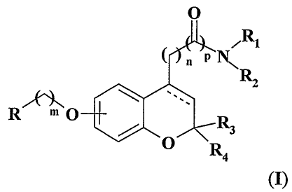

is a substituent in position 6 or 7 wherein:
R is an aromatic mono- or bi-cyclic carbocyclic ring or a mono- or bi-cyclic heterocyclic ring radical, said rings being optionally substituted by one or two substituents selected from (C₁-C₅) straight or branched alkyl, (C₁-C₅) straight or branched alkoxy, hydroxy, halogen and trifluoromethyl; m is zero or an integer from 1 to 3; n, p, R₁ and R₂ are as herein indicated and R₃ and R₄ are both hydrogen or taken together represent an oxygen atom, and the pharmaceutically acceptable salts thereof.

The compounds that are active as selective and reversible MAO-B inhibitors in vitro and in vivo, are useful as medicaments for the prevention and the treatment of CNS degenerative disorders.

This invention is related to novel aminoalkyl- and amidoalkyl-benzopyran derivatives of the following general formula (I)

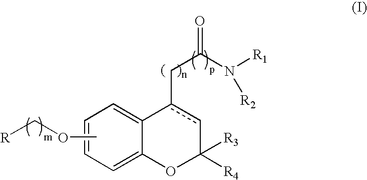

wherein:

the group

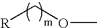

is a substituent in position 6 or 7 wherein:

R is a mono- or bi-cyclic (C₆-C₁₀) aryl radical or a mono- or bi-cyclic (5-10) membered heteroaryl radical, said radicals being optionally substituted by one or two substituents selected from (C₁-C₅) straight or branched alkyl, (C₁-C₅) straight or branched alkoxy, hydroxy, halo and trifluoromethyl;

m is zero or an integer from 1 to 3;

R₁ and R₂ each independently represent:

hydrogen;

(C₁-C₅) straight or branched alkyl optionally substituted by phenyl, where the phenyl group is optionally substituted by one or two substituents selected from (C₁-C₅) straight or branched alkyl, hydroxy, (C₁-C₅) straight or branched alkoxy, halo and trifluoromethyl;

(C₂-C₅) straight or branched alkyl substituted by amino;

phenyl, where the phenyl group is optionally substituted by one or two substituents selected from (C₁-C₅) straight or branched alkyl, hydroxy, (C₁-C₅) straight or branched alkoxy, halo and trifluoromethyl;

amino, (C₁-C₅) straight or branched alkyl- or dialkyl-amino;

or R₁ and R₂, taken together with the adjacent nitrogen atom form a saturated 5 to 7 member heterocyclic ring optionally containing one or two additional heteroatoms or groups selected from O, S and NR₅, wherein R₅ is hydrogen or a (C₁-C₅) straight or branched alkyl;

n is an integer from 1 to 3;

p is zero or 1;

R₃ and R₄ are both hydrogen, or taken together represent an oxygen atom;

the dotted line indicates nil or an additional bond;

with the proviso that:
(i) when R, m, n, p, R₃, R₄ and the dotted line are as above and one of R₁ and R₂ represents amino or (C₁-C₅) straight or branched alkylamino, then the other represents hydrogen or (C₁-C₅) straight or branched alkyl group;
(ii) when m and the dotted line are as above, n is 1, p is zero, R is a mono- or bi-cyclic (C₆-C₁₀) aryl radical optionally substituted as indicated above, R₃ and R₄ are both hydrogen, and one of R₁ and R₂ is hydrogen or (C₁-C₅) straight or branched alkyl, then the other may not be a (C₂-C₅) straight or branched alkyl substituted with phenyl where the phenyl group may be optionally substituted by one or two substituents as defined above;
(iii) when m is an integer form 1 to 3, n, p are as defined above, the dotted line indicates an additional bond; and

R₁ and R₂ each independently represent:

hydrogen;

(C₁-C₅) straight or branched alkyl optionally substituted by phenyl, where the phenyl group is optionally substituted by one or two substituents selected from (C₁-C₅) straight or branched alkyl, hydroxy, (C₁-C₅) straight or branched alkoxy, halo and trifluoromethyl;

(C₂-C₅) straight or branched alkyl substituted by amino;

phenyl, where the phenyl group is optionally substituted by one or two substituents selected from (C₁-C₅) straight or branched alkyl, hydroxy, (C₁-C₅) straight or branched alkoxy, halo and trifluoromethyl;

or, when p is zero, R₁ and R₂, taken together with the adjacent nitrogen atom form a saturated 5 to 6 member heterocyclic ring optionally containing one additional heteroatom or group selected from O, S and NR₅, wherein R₅ is hydrogen or a (C₁-C₅) straight or branched alkyl; and

R₃ and R₄ taken together represent an oxygen atom; and

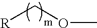

is a substituent in position 7;
then R cannot represent an unsubstituted mono- or bi-cyclic (C₆-C₁₀) aryl radical; if the case, either as single optical isomers or mixtures thereof, and the pharmaceutically acceptable salts and the pro-drugs thereof.

The invention includes the process for the preparation of the compounds of formula (I) and the pharmaceutical formulations containing them for use as medicament for the prevention and the treatment of CNS degenerative disorders, that are active as selective and reversible MAO-B inhibitors in vitro and in vivo.

### BACKGROUND OF THE INVENTION

Chemical Background

The term “benzopyran derivatives” as intended in this description and claims includes chroman and 2H-chromene compounds as well as the corresponding 2-oxo derivatives, i.e. chroman-2-one and 2H-chromen-2-one (coumarin) derivatives.

U.S. Pat. No. 5,554,611 (corresponding to EP 0655242 A) discloses coumarin derivatives for controlling and preventing disorders which arise from an elevated NO level, in particular, pathological decrease in blood pressure, as occurs in association with septic or haemorrhagic shock, in association with tumour therapy using cytokines, or in association with liver cirrhosis; inflammatory diseases, such as rheumatoid arthritis and, in particular, ulcerative colitis; insulin-dependent diabetes mellitus; transplant rejection reactions; arteriosclerosis; post-ischaemic tissue damage; reperfusion damage; myocarditis following infection with coxsackie virus; cardiomyopathy; forms of neuritis; encephalomyelitis; viral neurodegenerative diseases; Alzheimer's disease; hyperalgesia; epilepsy; migraine; acute kidney failure and glomerulonephritis; treatments in the stomach and uterus/placenta spheres and sperm motility.

U.S. Pat. No. 5,227,392 (corresponding to EP 0363796 A) and U.S. Pat. No. 5,100,914 disclose coumarin derivatives, with MAO-B inhibitory activity, wherein the substituents on the pyran ring do not contain either an amido or an amino group.

M. D. Ennis et al. in Bioorganic & Medicinal Chemistry Letters, 1993, 3, 1131-1136, describe the preparation of 4-aminomethyl-chroman derivatives active on 5-HT_{1A} or D-2 receptors wherein the benzene ring bears a methoxy substituent.

U.S. Pat. No. 4,977,166 discloses benzopyran derivatives having antiarrhythmic and antifibrillatory properties, wherein the alkoxy radical which may be positioned on the benzene ring does not contemplate the substitution with aromatic mono- or bi-cyclic (C₆-C₁₀) aryl radical or mono- or a bi-cyclic (5-10) membered heteroaryl radical.

WO 89/06534 discloses chroman and thiochroman compounds active as α-2 adrenergic antagonists wherein the substituents on the benzene ring do not contain a mono- or bi-cyclic (C₆-C₁₀) aryl radical or a mono- or bi-cyclic (5-10) membered heteroaryl radical.

U.S. Pat. No. 4,659,737 discloses N-substituted α-aminomethyl benzopyran derivatives having anti-hypertensive activities.

U.S. Pat. No. 4,486,428 discloses bicyclic benzo-fused compounds useful as analgesics, tranquilizers, antiemetic agents, diuretics, anticonvulsants, antidiarrheals, antitussives and in the treatment of glaucoma. Said benzo-fused compounds comprise benzopyran derivatives bearing two substituents in the positions 5 and 7.

EP-1318140 A discloses C5a receptor antagonist compounds which have an amido group directly bound to the position 4 of the pyran ring.

R. A. Geemon et al. in J. Med. Chem., 1982, 25, 393-397, describe the preparation of 6-methoxy-4-aminomethyl-chromene and -chroman derivatives and their interaction with serotonin receptors.

Biological Background

Monoamine oxidase (MAO) is an integral protein of the outer mitochondrial membrane and plays a major role in the in vivo inactivation of biogenic and diet-derived amines in both the CNS and in peripheral neurons and tissues. Two MAO enzymes are distinguished on the basis of their substrate preferences and sensitivity to inhibition by the MAO inhibitor clorgyline:
MAO type A (MAO-A) in the human CNS is responsible for the deamination of serotonin and noradrenaline. The highest MAO-A concentrations are in the catecholaminergic neurons of the locus ceruleus;
MAO type B (MAO-B) is responsible mainly for the catabolism of dopamine (DA). In contrast to the rat brain, MAO-B is the major isoform in the human and guinea pig CNS. The highest MAO-B concentrations are found in the serotoninergic neurons of the raphe nucleus and posterior hypothalamus. The nigral MAO-B is located primarily in glial cells.

MAO-B (but not MAO-A) activity in CNS increases with age in both humans and animals, perhaps as a result of the glial cell proliferation associated with neuronal loss. Increased MAO-B levels in Alzheimer's plaques have also been reported. Increased blood platelet MAO-B activity has been reported in both Alzheimer (AD) and Parkinson Disease (PD). MAO-B activity was reduced by 40% in the brain of smokers: tobacco smoking is associated with a reduced risk for PD.

Most currently investigated MAO-B inhibitors are irreversible inhibitors. The inhibition is very persistent (weeks), as its effect can only be overcome by de novo synthesis of the enzyme. Interest in the MAO-B inhibition was initially stimulated by the desire to elevate the reduced striatal DA concentration characteristic of PD, as increased DA concentration in the synaptic cleft would be expected as the primary effect of treatment with a selective MAO-B inhibitor. In PD, the need to supply the DA precursor L-Dopa, the golden standard in PD therapy, should thus be reduced. This is desirable, as L-Dopa, despite the excellent initial improvement achieved, is associated in the long term treatments with increasing severe side effects, including motor fluctuations, dyskinesia and dystonia.

In addition to the loss of cholinergic neurons, there is a decrease in the levels of the DA, noradrenaline and serotonin in the brain of AD patients. MAO-B inhibitors may act both, by reducing the formation of oxygen radicals and preventing the breakdown of monoamines and thus elevating their level in the brain of AD patients.

The compounds of this invention are useful in all the pathologies deriving from neurodegenerative processes and/or oxidative metabolic stress and/or lack of biogenic amines, for example Parkinson's disease, movement disorders (e.g. postencephalitic parkinsonism, progressive supranuclear palsy, corticobasal degeneration), restless leg syndrome, epilepsy, Alzheimer's disease and other dementias such as senile dementia of the Parkinson's type, vascular dementia and Lewy body dementia, amyotrophic lateral sclerosis, Down's syndrome, Huntington's disease, stroke, ischemia, CNS trauma. They are also useful for the treatment of narcolepsy, Tourette's syndrome, attention deficit hyperactivity disorders, negative symptoms of schizophrenia, drug addiction, smoking cessation and obesity.

There are evidences in the literature that demonstrate the potential therapeutic benefits of MAO-B inhibitors as can be seen in the following references: P. H. Wender J. Clin. Psychiatry, 1998, 59, 76-87; E. J. Houtsmuller et al. Psychopharmacology, 2004, 172, 31; J. E. Rose et al. Nicot. Tob. Res., 2001, 3, 383-388; P. Riederer et al. Curr. Med. Chem., 2004, 11, 2033-43; P. Jenner Neurology 2004, 63, S13-22; P. H. Yu Gen. Pharmacol., 1994, 25, 1527-39; M. Yamada Neurotoxicology, 2004, 25, 215-21; J. C. Delumeau J. Neural. Transm. Suppl. 1994, 41, 259-66.

### DESCRIPTION OF THE INVENTION

This invention is related to novel aminoalkyl- and amidoalkyl-benzopyran derivatives of the following general formula (I)

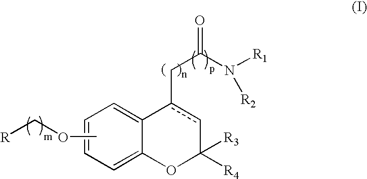

wherein:

the group

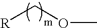

is a substituent in position 6 or 7 wherein:

R is a mono- or bi-cyclic (C₆-C₁₀) aryl radical or a mono- or bi-cyclic (5-10) membered heteroaryl radical, said radicals being optionally substituted by one or two substituents selected from (C₁-C₅) straight or branched alkyl, (C₁-C₅) straight or branched alkoxy, hydroxy, halo and trifluoromethyl;

m is zero or an integer from 1 to 3;

R₁ and R₂ each independently represent:

hydrogen;

(C₁-C₅) straight or branched alkyl optionally substituted by phenyl, where the phenyl group is optionally substituted by one or two substituents selected from (C₁-C₅) straight or branched alkyl, hydroxy, (C₁-C₅) straight or branched alkoxy, halo and trifluoromethyl;

(C₂-C₅) straight or branched alkyl substituted by amino;

phenyl, where the phenyl group is optionally substituted by one or two substituents selected from (C₁-C₅) straight or branched alkyl, hydroxy, (C₁-C₅) straight or branched alkoxy, halo and trifluoromethyl;

amino, (C₁-C₅) straight or branched alkyl- or dialkyl-amino;

or R₁ and R₂, taken together with the adjacent nitrogen atom form a saturated 5 to 7 member heterocyclic ring optionally containing one or two additional heteroatoms or groups selected from O, S and NR₅, wherein R₅ is hydrogen or a (C₁-C₅) straight or branched alkyl;

n is an integer from 1 to 3;

p is zero or 1;

R₃ and R₄ are both hydrogen, or taken together represent an oxygen atom;

the dotted line indicates nil or an additional bond;

with the proviso that:
(i) when R, m, n, p, R₃, R₄ and the dotted line are as above and one of R₁ and R₂ represents amino or (C₁-C₅) straight or branched alkylamino, then the other represents hydrogen or (C₁-C₅) straight or branched alkyl group;
(ii) when in and the dotted line are as above, n is 1, p is zero, R is a mono- or bi-cyclic (C₆-C₁₀) aryl radical optionally substituted as indicated above, R₃ and R₄ are both hydrogen, and one of R₁ and R₂ is hydrogen or (C₁-C₅) straight or branched alkyl, then the other may not be a (C₂-C₅) straight or branched alkyl substituted with phenyl where the phenyl group may be optionally substituted by one or two substituents as defined above;
(iii) when m is an integer from 1 to 3, n, p are as defined above, the dotted line indicates an additional bond; and

R₁ and R₂ each independently represent:

hydrogen;

(C₁-C₅) straight or branched alkyl optionally substituted by phenyl, where the phenyl group is optionally substituted by one or two substituents selected from (C₁-C₅) straight or branched alkyl, hydroxy, (C₁-C₅) straight or branched alkoxy, halo and trifluoromethyl;

(C₂-C₅) straight or branched alkyl substituted by amino;

phenyl, where the phenyl group is optionally substituted by one or two substituents selected from (C₁-C₅) straight or branched alkyl, hydroxy, (C₁-C₅) straight or branched alkoxy, halo and trifluoromethyl;

or, when p is zero, R₁ and R₂, taken together with the adjacent nitrogen atom form a saturated 5 to 6 member heterocyclic ring optionally containing one additional heteroatom or group selected from O, S and NR₅, wherein R₅ is hydrogen or a (C₁-C₅) straight or branched alkyl; and

R₃ and R₄ taken together represent an oxygen atom; and

the group

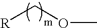

is a substituent in position 7;
then R cannot represent an unsubstituted mono- or bi-cyclic (C₆-C₁₀) aryl radical; if the case, either as single optical isomers or mixtures thereof, and the pharmaceutically acceptable salts and the pro-drugs thereof.

The invention includes the process for the preparation of the compounds of formula (I) and the same compounds and the pharmaceutical formulations containing them for use as medicament for the prevention and the treatment of CNS degenerative disorders, that are active as selective and reversible MAO-B inhibitors in vitro and in vivo.

According to this description and claims, a “mono- or bi-cyclic (C₆-C₁₀) aryl radical” is a radical derived from a mono- or by-cyclic aromatic ring system of, respectively, 6, 9 or 10 carbon atoms such as benzene, indene and naphthalene and includes also indan and tetrahydronaphtalene.

A “mono- or bi-cyclic (5-10) membered heteroaryl radical” is a radical derived from a mono- or by-cyclic heteroaromatic ring system of, respectively, 5 6, 9 or 10 members which contains one or two heteroatoms selected from N, O and S. Examples of said radicals are: furyl, thienyl, pyrrolyl, imidazolyl, pyridyl, indolyl, isoindolyl, quinolyl, isoquinolyl, benzofuranyl, and benzopyranyl.

The term “halo” indicate chloro, fluoro, bromo or iodio, preferably, chloro, fluoro or bromo, more preferably, chloro or fluoro.

The optional substituents in the above defined “aryl” and “heteroaryl” radicals represented by the symbol R and in the phenyl groups, when they are present in R₁ and/or R₂, may be in any position. The pharmaceutically acceptable salts of the compounds of formula (I) include acid addition salts with inorganic acids, e.g. hydrochloric, hydrobromic, sulphuric, and phosphoric or organic acids, e.g. acetic, propionic, benzoic, cinnamic, mandelic, salicylic, glycolic, lactic, oxalic, malic, maleic, malonic, fumaric, tartaric, citric, p. toluenesulfonic, methanesulfonic acid and the like.

According to an aspect of this invention, a group of preferred compounds of formula (I) as defined above is represented by the compounds of formula (I) wherein:

R is phenyl substituted by one or two substituents selected from (C₁-C₄) straight or branched alkyl, (C₁-C₄) straight or branched alkoxy, halo, and trifluoromethyl, or R is pyridyl;

m is zero, 1 or 2;

R₁ and R₂ each independently represents hydrogen, (C₁-C₄) straight or branched alkyl or phenyl-(C₁-C₂) alkyl; or one of R₁ and R₂ represents amino and the other represents hydrogen or (C₁-C₄) straight or branched alkyl; or R₁ and R₂ taken together with the adjacent nitrogen atom form a saturated 5 to 6 membered heterocyclic ring optionally containing an additional heteroatom selected from O, S and N(C₁-C₄) straight or branched alkyl.

n is 1, 2 or 3;

p is zero or 1;

R₃ and R₄ taken together represent an oxygen atom;

the dotted line indicates nil or an additional bond;

According to a further aspect of this invention, a group of most preferred compounds of formula (I) as defined above, is represented by the compounds of formula (I) wherein:

R is phenyl substituted by one substituent selected from (C₁-C₃) straight or branched alkyl, (C₁-C₃) straight or branched alkoxy, fluoro, chloro, and trifluoromethyl, or R is pyridyl;

m is 1;

R₁ and R₂ each independently represent hydrogen, (C₁-C₃) straight or branched alkyl or benzyl; or one of R₁ and R₂ represents amino and the other represents hydrogen or (C₁-C₃) straight or branched alkyl; or R₁ and R₂ taken together with the adjacent nitrogen atom form a saturated 5 to 6 membered heterocyclic ring containing one additional heteroatom selected from O, S and N(C₁-C₃) straight or branched alkyl.

n is 1 or 2;

p is zero or 1;

R₃ and R₄ taken together represents an oxygen atom;

the dotted line indicates an additional bond;

According to another aspect of this invention, a group of preferred compounds of formula (I) as defined above is represented by the compounds of formula (I) wherein:

R is phenyl optionally substituted by one or two substituents selected from (C₁-C₄) straight or branched alkyl, (C₁-C₄) straight or branched alkoxy, halo and trifluoromethyl, or R is pyridyl;

m is zero, 1 or 2;

R₁ and R₂ each independently represents hydrogen, (C₁-C₄) straight or branched alkyl or benzyl; or one of R₁ and R₂ represents amino and the other represents hydrogen or (C₁-C₄) straight or branched alkyl; or R₁ and R₂ taken together with the adjacent nitrogen atom form a saturated 5 to 6 membered heterocyclic ring optionally containing an additional heteroatom selected from O, S, and N(C₁-C₃) straight or branched alkyl.

n is 1, 2 or 3;

p is zero or 1;

R₃ and R₄ are both hydrogen;

the dotted line indicates nil or an additional bond;

According to a further aspect of this invention, a group of most preferred compounds of formula (I) as defined above, is represented by the compounds of formula (I) wherein:

R is phenyl optionally substituted by one substituent selected from (C₁-C₃) straight or branched alkyl, (C₁-C₃) straight or branched alkoxy, fluoro, chloro and trifluoromethyl, or R is pyridyl;

m is 1;

R₁ and R₂ each independently represent hydrogen or (C₁-C₃) straight or branched alkyl or benzyl; or one of R₁ and R₂ represent amino and the other represents hydrogen or (C₁-C₃) straight or branched alkyl; or R₁ and R₂ taken together with the adjacent nitrogen atom form a saturated 5 to 6 membered heterocyclic ring containing one additional heteroatom selected from O, S and N(C₁-C₃) straight or branched alkyl.

n is 1 or 2;

p is zero or 1;

R₃ and R₄ are both hydrogen;

the dotted line indicates nil or an additional bond;

Examples of specific compounds of the invention are:
4-[(Hydrazinocarbonyl)methyl]-7-benzyloxy-2H-chromen-2-one;
4-[(Aminocarbonyl)methyl]-7-(3-hydroxybenzyloxy)-2H-chromen-2-one;
4-[(Aminocarbonyl)methyl]-7-(pyridin-3-yl)methoxy-2H-chromen-2-one;
4-[(Aminocarbonyl)methyl]-7-(pyridin-4-yl)methoxy-2H-chromen-2-one;
4-[(Aminocarbonyl)methyl]-7-(3-chlorobenzyloxy)-2H-chromen-2-one;
4-[(Hydrazinocarbonyl)methyl]-6-(3-chlorobenzyloxy)-2H-chromen-2-one;
4-[(Hydrazinocarbonyl)methyl]-7-(3-chlorobenzyloxy)-2H-chromen-2-one;
4-[(Methylaminocarbonyl)methyl]-7-(3-chlorobenzyloxy)-2H-chromen-2-one;
4-[(Butylaminocarbonyl)methyl]-7-(3-chlorobenzyloxy)-2H-chromen-2-one;
4-[(Benzylaminocarbonyl)methyl]-7-(3-chlorobenzyloxy)-2H-chromen-2-one;
4-[(Dimethylaminocarbonyl)methyl]-7-(3-chlorobenzyloxy)-2H-chromen-2-one;
4-[(N-Butyl-N-methylaminocarbonyl)methyl]-7-(3-chlorobenzyloxy)-2H-chromen-2-one;
4-[[(2-Aminoethyl)aminocarbonyl]methyl]-7-(3-chlorobenzyloxy)-2H-chromen-2-one;
4-[(Aminocarbonyl)methyl]-6-(3-fluorobenzyloxy)-2H-chromen-2-one;
4-[(Aminocarbonyl)methyl]-7-(3-fluorobenzyloxy)-2H-chromen-2-one;
4-[(Hydrazinocarbonyl)methyl]-7-(3-fluorobenzyloxy)-2H-chromen-2-one;
4-[(Methylaminocarbonyl)methyl]-7-(3-fluorobenzyloxy)-2H-chromen-2-one;
4-[(Benzylaminocarbonyl)methyl]-7-(3-fluorobenzyloxy)-2H-chromen-2-one;
4-[(Dimethylaminocarbonyl)methyl]-7-(3-fluorobenzyloxy)-2H-chromen-2-one;
4-[2-(Hydrazinocarbonyl)ethyl]-7-benzyloxy-2H-chromen-2-one;
4-[2-(Aminocarbonyl)ethyl]-7-(3-chlorobenzyloxy)-2H-chromen-2-one;
4-[2-(Hydrazinocarbonyl)ethyl]-7-(3-chlorobenzyloxy)-2H-chromen-2-one;
4-[2-(Methylaminocarbonyl)ethyl]-7-(3-chlorobenzyloxy)-2H-chromen-2-one;
4-[2-(Benzylaminocarbonyl)ethyl]-6-(3-chlorobenzyloxy)-2H-chromen-2-one;
4-[2-(Benzylaminocarbonyl)ethyl]-7-(3-chlorobenzyloxy)-2H-chromen-2-one;
4-[2-(Dimethylaminocarbonyl)ethyl]-7-(3-chlorobenzyloxy)-2H-chromen-2-one;
4-[2-(Aminocarbonyl)ethyl]-7-(3-fluorobenzyloxy)-2H-chromen-2-one;
4-[2-(Hydrazinocarbonyl)ethyl]-7-(3-fluorobenzyloxy)-2H-chromen-2-one;
4-[2-(Methylaminocarbonyl)ethyl]-7-(3-fluorobenzyloxy)-2H-chromen-2-one;
4-[2-(Butylaminocarbonyl)ethyl]-7-(3-fluorobenzyloxy)-2H-chromen-2-one;
4-[2-(Benzylaminocarbonyl)ethyl]-7-(3-fluorobenzyloxy)-2H-chromen-2-one;
4-[2-(Dimethylaminocarbonyl)ethyl]-7-(3-fluorobenzyloxy)-2H-chromen-2-one;
4-[(Aminocarbonyl)methyl]-7-benzyloxy-2H-chromene;
4-[(Hydrazinocarbonyl)methyl]-7-benzyloxy-2H-chromene;
4-[(Methylaminocarbonyl)methyl]-7-benzyloxy-2H-chromene;
4-[(Dimethylaminocarbonyl)methyl]-7-benzyloxy-2H-chromene;
4-[(Dimethylaminocarbonyl)methyl]-7-(3-chlorobenzyloxy)-2H-chromene;
4-[(Aminocarbonyl)methyl]-7-(3-fluorobenzyloxy)-2H-chromene;
4-[(Hydrazinocarbonyl)methyl]-7-(3-fluorobenzyloxy)-2H-chromene;
4-[(Methylaminocarbonyl)methyl]-7-(3-fluorobenzyloxy)-2H-chromene;
4-[(Dimethylaminocarbonyl)methyl]-7-(3-fluorobenzyloxy)-2H-chromene;
4-[2-(Aminocarbonyl)ethyl]-6-benzyloxy-2H-chromene;
4-[2-(Aminocarbonyl)ethyl]-7-benzyloxy-2H-chromene;
4-[2-(Hydrazinocarbonyl)ethyl]-7-benzyloxy-2H-chromene;
4-[2-(Methylaminocarbonyl)ethyl]-7-benzyloxy-2H-chromene;
4-[2-(Dimethylaminocarbonyl)ethyl]-7-benzyloxy-2H-chromene;
4-[2-(Aminocarbonyl)ethyl]-7-(3-fluorobenzyloxy)-2H-chromene;
4-[2-(Hydrazinocarbonyl)ethyl]-7-(3-fluorobenzyloxy)-2H-chromene;
4-[2-(Methylaminocarbonyl)ethyl]-7-(3-fluorobenzyloxy)-2H-chromene;
4-[(Aminocarbonyl)methyl]-7-benzyloxy-chroman;
4-[(Hydrazinocarbonyl)methyl]-7-benzyloxy-chroman;
4-[(Methylaminocarbonyl)methyl]-7-benzyloxy-chroman;
4-[(Aminocarbonyl)methyl]-7-(3-fluorobenzyloxy)-chroman;
4-[(Hydrazinocarbonyl)methyl]-7-(3-fluorobenzyloxy)-chroman;
4-[(Methylaminocarbonyl)methyl]-7-(3-fluorobenzyloxy)-chroman;
4-[2-(Hydrazinocarbonyl)ethyl]-7-benzyloxy-chroman;
4-[2-(Methylaminocarbonyl)ethyl]-7-benzyloxy-chroman;
4-[2-(Aminocarbonyl)ethyl]-7-(3-fluorobenzyloxy)-chroman;
4-[2-(Methylaminocarbonyl)ethyl]-7-(3-fluorobenzyloxy)-chroman;
4-Aminomethyl-7-(3-chlorobenzyloxy)-2H-chromen-2-one;
4-(2-Aminoethyl)-7-(3-chlorobenzyloxy)-2H-chromen-2-one;
4-[(Methylamino)methyl]-7-(3-chlorobenzyloxy)-2H-chromen-2-one;
4-[(Dimethylamino)methyl]-6-(3-chlorobenzyloxy)-2H-chromen-2-one;
4-[(Dimethylamino)methyl]-7-(3-chlorobenzyloxy)-2H-chromen-2-one;
4-[2-(Methylamino)ethyl]-7-(3-chlorobenzyloxy)-2H-chromen-2-one;
4-[(Ethylamino)methyl]-7-(3-chlorobenzyloxy)-2H-chromen-2-one;
4-[2-(Ethylamino)ethyl]-7-(3-chlorobenzyloxy)-2H-chromen-2-one;
4-[(Benzylamino)methyl]-7-(3-chlorobenzyloxy)-2H-chromen-2-one;
4-[(N-Benzyl-N-methylamino)methyl]-7-(3-chlorobenzyloxy)-2H-chromen-2-one;
4-Aminomethyl-7-(3-fluorobenzyloxy)-2H-chromen-2-one;
4-[(Methylamino)methyl]-7-(3-fluorobenzyloxy)-2H-chromen-2-one;
4-[(Dimethylamino)methyl]-7-(3-fluorobenzyloxy)-2H-chromen-2-one;
4-[2-(Methylamino)ethyl]-7-(3-fluorobenzyloxy)-2H-chromen-2-one;
4-[(Ethylamino)methyl]-7-(3-fluorobenzyloxy)-2H-chromen-2-one;
4-[(Isopropylamino)methyl]-7-(3-fluorobenzyloxy)-2H-chromen-2-one;
4-(2-Aminoethyl)-7-benzyloxy-2H-chromene;
4-[2-(Methylamino)ethyl]-7-benzyloxy-2H-chromene;
4-(2-Aminoethyl)-7-(3-chlorobenzyloxy)-2H-chromene;
4-(2-Aminoethyl)-7-(3-fluorobenzyloxy)-2H-chromene;
4-(2-Aminoethyl)-6-benzyloxy-chroman;
4-(2-Aminoethyl)-7-benzyloxy-chroman;
4-(3-Aminopropyl)-7-benzyloxy-chroman;
4-[(Methylamino)methyl]-7-benzyloxy-chroman;
4-[2-(Methylamino)ethyl]-7-benzyloxy-chroman;
4-[3-(Methylamino)propyl]-7-benzyloxy-chroman;
4-Aminomethyl-7-(3-chlorobenzyloxy)-chroman;
4-(2-Aminoethyl)-7-(3-chlorobenzyloxy)-chroman;
4-[(Methylamino)methyl]-7-(3-chlorobenzyloxy)-chroman;
4-Aminomethyl-7-(3-fluorobenzyloxy)-chroman;
4-(2-Aminoethyl)-7-(3-fluorobenzyloxy)-chroman;
4-[(Methylamino)methyl]-7-(3-fluorobenzyloxy)-chroman;
4-[2-(Methylamino)ethyl]-7-(3-fluorobenzyloxy)-chroman;
   and the pharmaceutically acceptable salts and the pro-drugs thereof.

Where the compounds of this invention contain asymmetric carbon atoms and, therefore, they can exist as single optical isomers or a mixture thereof (e.g. in all cases where the dotted line in formula (I) does not indicate an additional bond or where a branched alkyl moiety contains an asymmetric carbon atom), the invention includes within its scope all the possible optical isomers of said compounds and the mixtures thereof.

The compounds of the invention can be prepared by different methods.

The aminomethyl-coumarin derivatives (aminomethyl-2H-chromene-2-one derivatives) are prepared starting from 4-(chloromethyl)-(6 or 7)-hydroxy-2H-chromen-2-one, which can be obtained from ethyl 4-chloroacetoacetate and resorcinol or hydroquinone through the classical von Pechmann procedure (H. Von Pechmann; C. Duisberg, Chem. Ber., 1883, 16, 2119-2128; N. Nguyen-Hai et al. Bioorganic & Medicinal Chemistry Letters, 2002, 12, 2345-2348), using catalytic amounts of sulphuric acid and heating the reaction mixture to 120° C. or, as an alternative procedure, by using sulphuric acid as a solvent, at temperatures ranging from −10° C. to 10° C.

The second step is the aryl- or heteroaryl-alkylation of 4-(chloromethyl)-(6 or 7)-hydroxy-2H-chromen-2-one with the appropriate aryl- or heteroaryl-alkyl bromide, in the presence of anhydrous K₂CO₃ in a refluxing absolute alcohol such as methanol or ethanol or propanol.

Primary amines were obtained through the synthesis of the intermediate azides, obtained by refluxing the different 6- or 7-arylalkoxy or 6- or 7-heteroarylalkoxy-4-chloromethyl-2H-chromen-2-one compounds with NaN₃ in a lower alkyl alcohol and reducing the azido derivatives with SnCl₂ (S. N. Maiti et al., Tetrahedron Letters, 1986, 13, 1423-1424) in methanol or ethanol.

The mono- and di-alkylamino derivatives were obtained by reacting the appropriate 6- or 7-arylalkoxy- or 6- or 7-heteroarylalkoxy-4-(chloromethyl)-2H-chromen-2-one derivatives with the commercially available, or very easily obtainable, solutions of the suitable primary or secondary amines, in THF at 40-65° C. or in refluxing lower alkyl anhydrous alcohol in the presence of a HCl scavenger as, for example, potassium carbonate.

The 4-aminocarbonylmethyl- and the 4-(2-aminoethyl)-coumarin compounds (and the 4-aminocarbonyl-(C₂-C₃)alkyl- and the 4-(3-aminopropyl)-alkyl-coumarin compounds), were prepared starting from resorcinol and diethyl-1,3-acetonedicarboxylate (and the corresponding homologues H₅C₂OOC—(CH₂)ₖ—CH₂—CO—CH₂—COOC₂H₅, wherein k is 1 or 2), according to the von Pechmann classical procedure (see above). The 4-[(ethoxycarbonyl)methyl]-(6- or 7-)hydroxy-2H-chromen-2-one (and the corresponding 4-[2-(ethoxycarbonyl)ethyl]- and 4-[3-(ethoxycarbonyl)propyl]-substituted homologues) obtained was reacted with ammonia or the appropriate amine at 50-100° C. for 20-60 hours to afford the corresponding 4-[(aminocarbonyl)methyl]-(6- or 7-)hydroxy-2H-chromen-2-one derivatives (and the corresponding 4-[2-(aminocarbonyl)ethyl]- and 4-[3-(aminocarbonyl)propyl]-homologues).

A Mitsunobu condensation of the “carbonyl” compounds with the appropriate

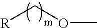

aryl or heteroaryl-substituted alcohol, gave the corresponding 6- or 7-ethers of the 4-[(aminocarbonyl)methyl]-2H-chromen-2-one derivatives. The corresponding primary 4-(2-aminoethyl)-coumarin and 4-(3-aminopropyl)-coumarin compounds could be best obtained by converting the corresponding 4-aminocarbonyl derivatives of formula (I), wherein n is 1 or 2, into nitriles with trifluoroacetic anhydride, according to a method developed by Carotti (A. Carotti et al. Tetrahedron Letters, 1977, 21, 1813-1816) and reduction of the nitriles with sodium borohydride in the presence of cobaltous chloride (T. Satoh et al. Tetrahedron Letters, 1969, 52, 4555-4558).

The other 4-mono- and 4-(di-substituted-2-aminoethyl- (or 3-aminopropyl-))-coumarin derivatives, were best obtained by reacting for 6-12 hours, the 4-(2-bromoethyl- (or 3-bromopropyl-))-2H-chromen-2-one 6- or 7-ether derivatives with the suitable primary or secondary amines, in aprotic solvents such as THF or acetone, or protic solvents such as lower alkyl alcohols, in the presence of KI and of an acid scavenger such as, for example, potassium carbonate or an excess of the reacting amines, at temperatures ranging from 30 to 70° C.

The 4-(2-bromoethyl)-2H-chromen-2-one 6- or 7-ether derivatives were obtained starting from 4-[(ethoxycarbonyl)methyl]-(6- or 7-)-hydroxy-2H-chromen-2-one compounds, which were hydrolyzed to the corresponding 4-carboxymethyl derivatives, reduced to the 4-(2-hydroxyethyl)-alcohols and brominated with CBr₄ and triphenyl phosphine in methylene chloride at 0-35° C. These 4-(2-bromoethyl)-(6- or 7-)hydroxy derivatives were then transformed into the appropriate 6- or 7-ethers. Analogous procedures were adopted for obtaining the 4-(3-bromopropyl)-substituted homologues.

All the reactions and reaction conditions cited in the paragraph here above are well known to those skilled in the art.

The 2H-chromene derivatives were obtained by selective reduction of the appropriate 2H-chromen-2-one compounds either with lithium aluminum hydride or diborane in aprotic anhydrous solvents such as THF, at temperature ranging from −20° C. to room temperature.

The chroman derivatives were obtained by selective reduction of the corresponding 2H-chromene compounds with Pd/H₂ (S. Maki, Tetrahedron Lett. 2003, 44, 3717-3721)

Pharmacology

The compounds of this invention are able to selectively and reversibly inhibit MAO-B in vitro and in vivo.

The compounds of the invention which are potent inhibitors of MAO-B (IC₅₀ in the submicromolar-nMolar range) have generally no relevant effect on MAO-A. The MAO-B inhibition is not time-dependent, which is characteristic of reversible inhibitors. After oral, single dose administration in mice, the compounds behave as potent and reversible, short-acting MAO-B inhibitors with full recovery of the MAO-B enzymatic activity 8-16 hours after the administration. Compounds of the invention are useful for the treatment of all conditions mediated by MAO-B enzymes.

It will be appreciated that the compounds of the invention may advantageously be used in conjunction with one or more other therapeutic agents. Examples of suitable agents for adjunctive therapy include L-Dopa and/or a dopamine agonist and/or a monoamine reuptake inhibitor; a catechol-O-methyltransferase inhibitor; a free radical scavenger; an adenosine A2 antagonist; a glutamate modulator, such as a glutamate release inhibitor or NMDA or AMPA antagonist; a nitric oxide synthase (NOS) inhibitor, such as an iNOS or an nNOS inhibitor; a sodium and/or calcium channel blocker; a serotonin receptor agonist; a substance P antagonist (e.g. an NK1 antagonist); an alfa-1 or alfa-2 adrenergic agonist; a nicotinic receptor agonist; an α-synuclein aggregation inhibitor; a cholinesterase inhibitor; a cholesterol lowering agent (such a simvastatin, lovastatin, atorvastatin); a β-secretase modulator; a β-amyloid aggregation inhibitor; a cannabinoid; gabapentin and related compounds; a tricyclic antidepressant (e.g. amitryptiline); a neuron stabilizing antiepileptic drug; a matrix metalloproteinase inhibitor; an inhibitor of the release of TNFα; an antibody therapy, such as monoclonal antibody therapy; an antiviral agent, such as a nucleoside inhibitor (e.g. lamivudine) or an immune system modulator (e.g. interferon); an analgesic, such as a cyclooxygenase-2 inhibitor; a local anaesthetic; a stimulant including caffeine; a decongestant (e.g. phenylephrine, phenylpropanolamine, pseudoephedrine, oxymetazoline, epinephrine, naphazoline); an antitussive (e.g. codeine, hydrocodone, carmiphen, carbetapentane, or dextramethorphan); a diuretic or a sedating or non-sedating antihistamine.

The compounds of the present invention are useful in human and veterinary medicine. It is to be understood that reference to treatment includes both treatments of established symptoms and prophylactic treatment, unless explicitly stated otherwise.

The compounds of this invention can be administered in conventional manner, e.g. orally, subcutaneously, intravenously, intramuscularly, intraperitoneally or transdermally. The dose is usually depending on the age, condition, weight of the patient and route of administration. In general, the practitioner will determine the dosage which he considers the more suitable as a function of the above factors specific to the subject to be treated. The dosages are generally between 1 mg and 1 g of active product per patient per day. The daily dose can be divided into several smaller doses, e.g. into 2 to 4 doses which are administered separately.

The derivatives of formula (I) as defined above can be administered as the “active ingredient” of a pharmaceutically acceptable composition, which can be prepared by conventional procedures, for instance by mixing the active ingredient with pharmaceutically acceptable, therapeutically inert organic and/or inorganic carrier materials.

The composition comprising the above defined derivatives can be administered by various routes, e.g. orally, in the form of tablets, troches, capsules, sugar or film coated tablets, liquid solutions, emulsions or suspensions; rectally, in the form of suppositories; parenterally, e.g. by intramuscular or intravenous injection or infusion; or transdermally in form of patch or gel or cream.

Suitable pharmaceutically acceptable, therapeutically inert organic and/or inorganic carrier materials useful in the preparation of such composition include, for example, water, gelatin, arabic gum, lactose, starch, cellulose, magnesium stearate, talc, vegetable oils, polyalkyleneglycols, cyclodextrin and the like. The compositions comprising the aminoalkyl-benzopyran derivatives of formula (I) as defined above can be sterilized and may contain further well known components, such as, for example, preservatives, stabilizers, wetting or emulsifying agents, e.g. paraffin oil, mannide monooleate, salts to adjust osmotic pressure, buffers and the like.

For example, the solid oral forms may contain, together with the active ingredient, diluents, e.g. lactose, dextrose, saccharose, cellulose, corn starch or potato starch; lubricants, e.g. silica, talc, stearic acid, magnesium or calcium stearate, and/or polyethylene glycols; binding agents, e.g. starches, arabic gum, gelatin, methylcellulose, carboxymethylcellulose or polyvinyl pyrrolidone; desegregating agents, e.g. a starch, alginic acid, alginates or sodium starch glycolate; effervescing mixtures; dyestuffs; sweeteners; wetting agents such as lecithin, polysorbates, laurylsulphates; and, in general, non-toxic and pharmacologically inactive substances used in pharmaceutical formulations. Said pharmaceutical preparations may be manufactured in known manner, for example, by means of mixing, granulating, tabletting, sugar-coating, or film-coating processes.

The oral formulations comprise sustained release formulations that can be prepared in conventional manner, for instance by applying an enteric coating to tablets and granules.

The liquid dispersion for oral administration may be e.g. syrups, emulsions or suspensions.

The syrups may contain as a carrier, for example, saccharose or saccharose with glycerine and/or mannitol and/or sorbitol.

Suspensions and emulsions may contain as a carrier, for example, a natural gum, agar, sodium alginate, pectin, methylcellulose, carboxymethylcellulose, or polyvinyl alcohol. The suspensions or solutions for intramuscular injections may contain, together with the active compound, a pharmaceutically acceptable carrier, e.g. sterile water, olive oil, ethyl oleate, glycols, e.g. propylene glycol, and, if desired, a suitable amount of lidocaine hydrochloride. The solutions for intravenous injections or infusion may contain as carrier, for example, sterile water or preferably they may be in the form of sterile, aqueous, isotonic saline solutions.

The suppositories may contain, together with the active ingredient, a pharmaceutically acceptable carrier, e.g. cocoa butter, polyethylene glycol, a polyoxyethylene sorbitan fatty acid ester surfactants or lecithin.

The composition comprising the aminoalkyl-benzopyran derivatives of formula (I) as above defined is generally in the form of a dose unit containing, for example, from 1 mg to 500 mg of active ingredient, most preferably from 1 to 100 mg.

Optimal therapeutically effective doses to be administered may be readily determined by those skilled in the art and will vary, basically, with the strength of the preparation, with the mode of administration and with the advancement of the condition or disorder treated. In addition, factors associated with the specific patient being treated, including patient age, weight, diet and time of administration, will result in the need to adjust the dose to an appropriate therapeutically effective level.

### EXAMPLES

### Example 1

### 4-[(Dimethylaminocarbonyl)methyl]-7-benzyloxy-2H-chromene

To a solution of 4-[(dimethylaminocarbonyl)methyl]-7-benzyloxy-2H-chromen-2-one (0.067 g, 0.2 mmol) in 4 ml of anhydrous THF, LiAlH₄ (0.016 g, 0.42 mmol), was added portionwise over 1 hour. The mixture was stirred at room temperature for 6 hours. The excess LiAlH₄ was decomposed by careful addition of ethyl acetate and the mixture was filtered on celite. The solvent was evaporated under vacuum to give an oil that was purified by column chromatography on silica gel (eluant CHCl₃/MeOH 9.5/0.5 v/v).

Yield: 25%.

Yellow oil.

¹H-NMR (CDCl₃) δ: 7.44-7.32 (m, 5H); 6.79 (d, J=8.2, 1H); 6.63 (d, J=2.5, 1H); 6.54 (dd, J=8.2, J=2.5, 1H); 5.93 (t, J=7.0, 1H); 5.02 (s, 2H); 3.86 (d, J=7.0, 2H); 3.37 (s, 2H); 3.00 (s, 3H); 2.93 (s, 3H).

### Example 2

### 4-[(Aminocarbonyl)methyl]-7-(3-hydroxybenzyloxy)-2H-chromen-2-one

A solution of 0.43 g (1.95 mmol) of 4-[(aminocarbonyl)methyl]-7-hydroxy-2H-chromen-2-one, 5.7 g (19.5 mmol) (3-benzoyloxy)benzyl bromide and 2.5 g (19.5 mmol) of diisopropylethylamine in 50 ml of anhydrous THF, was stirred at 70° C. for 2 hours. The mixture was then cooled to room temperature, the solid which formed was filtered off, 1.5 ml of saturated methanolic sodium methylate solution was added and the whole mixture was stirred for 4 hours. After evaporation of the solvent under vacuum, the residue was taken up in 30 ml of ethyl acetate and 5 ml of 1 N HCl, the organic phase was separated, washed with brine and dried over anhydrous sodium sulphate. After evaporation of the solvent under vacuum, the yellow solid residue was purified by column chromatography on silica gel (eluant CH₂Cl₂/MeOH 8.5/1.5 v/v) to give the title compound in 30% yield.

Mp (dec.) 190-191° C.

¹H-NMR (DMSO-d₆) δ: 9.51 (s, 1H); 7.66-7.63 (m, 2H); 7.18-6.99 (m, 4H); 6.85-6.81 (m, 2H); 6.70-6.68 (m, 1H); 6.23 (s, 1H); 5.13 (s, 2H); 3.62 (s, 2H).

### Example 3

### 4-[(Hydrazinocarbonyl)methyl]-7-benzyloxy-2H-chromen-2-one

A solution of 0.025 g (0.06 mmol) of 4-[(tert-butoxycarbonylhydrazinocarbonyl)methyl]-7-benzyloxy-2H-chromen-2-one in 1 ml 1/1 mixture of CH₂Cl₂/CF₃COOH was stirred at room temperature for 20 minutes. The solvent was evaporated under vacuum and the oily residue was treated with diethyl ether to give a precipitate that was filtered and crystallized from ethanol.

Yield: 93%.

Mp: 164-165° C. dec.

¹H-NMR (DMSO-d₆) δ: 10.68 (b, 1H); 7.66 (d, J=8.8, 1H); 7.46-7.33 (m, 5H); 7.09 (d, J=2.2, 1H); 7.03 (dd, J=8.8, J=2.2, 1H); 6.29 (s, 1H); 5.22 (s, 2H); 3.78 (s, 4H).

### Example 4

### 4-[(Methylaminocarbonyl)methyl]-7-(3-chlorobenzyloxy)-2H-chromen-2-one

A sealed glass ampoule containing 0.730 g (2 mmol) of 4-[(ethoxycarbonyl)methyl]-7-(3-chlorobenzyloxy)-2H-chromen-2-one and 10 ml (20 mmol) of 2.0 M solution of methylamine in THF was placed in an oven at 90° C. for 60 hours. The solution was then evaporated under vacuum and the oily residue was purified by column chromatography on silica gel (eluant CHCl₃/MeOH 9.5/0.5 v/v) to give 349 mg (50%) of product with a melting point of 174-175° C.

¹H-NMR (DMSO-d₆) δ: 8.07 (b, 1H); 7.67 (d, J=8.8, 1H); 7.53 (s, 1H); 7.42-7.39 (m, 3H); 7.08-7.01 (m, 2H); 6.23 (s, 1H); 5.23 (s, 2H); 3.64 (s, 2H); 2.56 (s, 3H).

### Examples 5-9

The compounds of the following Examples 5-9 were obtained according to the same procedure described in Example 4 above by substituting methylamine with the appropriate amine.

### Example 5

### 4-[(Benzylaminocarbonyl)methyl]-7-(3-chlorobenzyloxy)-2H-chromen-2-one

Yield: 25%.

Mp: 170-171° C.

¹H-NMR (CDCl₃) δ: 7.60 (d, J=8.8, 1H); 7.43 (b, 1H); 7.37-7.26 (m, 6H); 7.18-7.15 (m, 2H); 6.92 (dd, J=8.8, J=2.5, 1H); 6.86 (d, J=2.5, 1H); 6.22 (s, 1H); 5.90 (b, 1H); 5.10 (s, 2H); 4.42 (d, J=5.8, 2H); 3.69 (s, 2H).

### Example 6

### 4-[(Butylaminocarbonyl)methyl]-7-(3-chlorobenzyloxy)-2H-chromen-2-one

Yield: 22%.

Mp: 112-113° C. from ethanol.

¹H-NMR (CDCl₃) δ: 7.60 (d, J=8.8, 1H); 7.43 (b, 1H); 7.34-7.30 (m, 3H); 6.91 (dd, J=8.8, J=2.5, 1H); 6.87 (d, J=2.5, 1H); 6.23 (s, 1H); 5.51 (b, 1H); 5.10 (s, 2H); 3.64 (s, 2H); 3.23 (q, J=6.7, 2H); 1.50-1.38 (m, 2H); 1.31-1.21 (m, 2H); 0.87 (t, J=7.2, 3H).

### Example 7

### 4-[(N-Butyl-N-methylaminocarbonyl)methyl]-7-(3-chlorobenzyloxy)-2H-chromen-2-one

Yield: 25%.

Oil.

¹H-NMR (CDCl₃) δ: 7.49 (d, J=8.8, 1H); 7.42 (b, 1H); 7.36-7.26 (m, 3H); 6.91 (d, J=8.8, 1H); 6.86 (s, 1H); 6.15 (s, 1H); 5.10 (s, 2H); 3.78 (s, 2H); 3.41 (t, J=7.4, 2H); 3.32 (t, J=7.4, 2H); 3.06 (s, 3H); 2.98 (s, 3H); 1.67-1.25 (m, 4H); 1.03-0.90 (m, 3H).

### Example 8

### 4-[Dimethylaminocarbonyl)methyl]-7-(3-chlorobenzyloxy)-2H-chromen-2-one

Yield: 62%.

Mp: 159-160° C.

¹H-NMR (CDCl₃) δ: 7.51 (d, J=8.8, 1H); 7.43 (b, 1H); 7.34-7.29 (m, 3H); 6.92 (dd, J=8.8, J=2.5, 1H); 6.87 (d, J=2.5, 1H); 6.14 (s, 1H); 5.10 (s, 2H); 3.79 (s, 2H); 3.10 (s, 3H); 3.02 (s, 3H).

### Example 9

### 4-[[(2-Aminoethyl)aminocarbonyl]methyl]-7-(3-chlorobenzyloxy)-2H-chromen-2-one

A solution of 0.03 g (0.06 mmol) of 4-[[(2-tert-butoxycarbonylaminoethyl)aminocarbonyl]methyl]-7-(3-chlorobenzyloxy)-2H-chromen-2-one in 1 ml of a 1/1 mixture of CH₂Cl₂/CF₃COOH was stirred at room temperature for 15 minutes. The solvent was evaporated under vacuum and the oily residue was treated with chloroform/n-hexane to give a pure solid.

Yield: 83%.

Mp: 144.5-145.5° C.

¹H-NMR (DMSO-d₆) δ: 8.33 (b, 1H); 7.74 (b, 2H, exchange with D₂O); 7.68 (d, J=8.8, 1H); 7.53 (s, 1H); 7.42-7.39 (m, 3H); 7.09 (d, J=2.5, 1H); 7.03 (dd, J=8.8, J=2.5, 1H); 6.25 (s, 1H); 5.23 (s, 2H); 3.69 (s, 2H); 3.28-3.26 (m, 2H); 2.83-2.81 (m, 2H).

### Example 10

### 4-Aminomethyl-7-(3-chlorobenzyloxy)-2H-chromen-2-one

To a clear solution of SnCl₂ dihydrate (664 mg, 3.5 mmol) in methanol (5 ml), 137 mg (0.4 mmol) of 4-azidomethyl-7-(3-chlorobenzyloxy)-2H-chromen-2-one were added over 1 hour in small portions. The mixture was stirred at room temperature for 3 hours. The solvent was evaporated under vacuum and the residue was poured into cold water. The pH was made strongly basic by addition of NaOH 3N and the resulting aqueous solution was extracted with ethyl acetate. The organic layers were collected, washed with brine, dried over anhydrous sodium sulphate and evaporated to dryness under vacuum. The resulting solid was purified by column chromatography on silica gel (eluant CHCl₃/CH₃OH (9.7/0.3 v/v) yielding 49.3 mg (39%) of a white solid with a 99% purity and a melting point of 166-167° C. (dec.).

ESI-MS m/z: [MNa]⁺=338.

¹H-NMR (DMSO-d₆) δ: 7.69 (d, 1H, J=8.8), 7.53 (s, 1H), 7.48-7.39 (m, 3H), 7.07 (d, 1H, J=2.5), 7.00 (dd, 1H, J=8.8, J=2.5), 6.39 (s, 1H), 5.23 (s, 2H), 3.90 (s, 2H).

### Example 11

### 4-Aminomethyl-7-(3-fluorobenzyloxy)-2H-chromen-2-one

This compound was prepared according to the same procedure of Example 10 by using 4-azidomethyl-7-(3-fluorobenzyloxy)-2H-chromen-2-one instead of 4-azidomethyl-7-(3-chlorobenzyoxy)-2H-chromen-2-one:

Yield: 50%.

ESI-MS m/z: [MNa]⁺=321.

¹H-NMR (DMSO-d₆) δ: 7.70 (d, 1H, J=8.8), 7.48-7.39 (m, 1H), 7.32-7.27 (m, 2H), 7.21-7.11 (m, 1H), 7.06 (d, 1H, J=2.5), 7.01 (dd, 1H, J=8.8, J=2.5), 6.41 (s, 1H), 5.25 (s, 2H), 3.91 (s, 2H).

### Example 12

### 4-[(Methylamino)methyl]-7-(3-chlorobenzyloxy)-2H-chromen-2-one

A mixture of 1.0 g (3.0 mmol) of 4-chloromethyl-7-(3-chlorobenzyloxy)-2H-chromen-2-one and 30 ml (60 mmol) of a 2M solution of methylamine in 30 ml of THF was stirred at 55° C. under argon for 8 hours. The mixture was cooled to room temperature and the inorganic precipitate was filtered off. The solvent was evaporated under vacuum and the resulting solid was purified by column chromatography on silica gel using AcOEt as eluant, yielding 276 mg (28%) of a pale yellow oil.

ESI-MS m/z: [MNa]⁺=352.

¹H-NMR (CDCl₃) δ: 7.60 (d, 1H, J=8.8), 7.43 (s, 1H), 7.34-7.31 (m, 3H), 6.92 (dd, 1H, J=8.8, J=2.8), 6.86 (d, 1H, J=2.8), 6.38 (s, 1H), 5.10 (s, 2H), 3.90 (s, 2H), 2.54 (s, 3H), 1.25 (s, 1H).

### Examples 13-17

The compounds of the following Examples 13-17 were prepared according to the same procedure described in Example 12 by substituting 4-chloromethyl-7-(3-chlorobenxyloxy)-2H-chromen-2-one and/or methylamine with the appropriate 2H-chromen-2-one and/or amine starting material.

### Example 13

### 4-[(Methylamino)methyl]-7-(3-fluorobenzyloxy)-2H-chromen-2-one

Yield: 22%.

Mp: 115-117° C.

ESI-MS m/z: [MNa]⁺=336.

¹H-NMR (DMSO-d₆) δ: 7.73 (d, 1H, J=8.8), 7.47-7.40 (m, 1H), 7.31-7.28 (m, 2H), 7.19-7.12 (m, 1H), 7.05 (d, 1H, J=2.5), 7.00 (dd, 1H, J=8.8, J=2.5), 6.29 (s, 1H), 5.23 (s, 2H), 3.81 (s, 2H), 2.32 (s, 3H).

### Example 14

### 4-[(Ethylamino)methyl]-7-(3-fluorobenzyloxy)-2H-chromen-2-one

¹H-NMR (DMSO-d₆) δ: 7.74 (d, 1H, J=8.8), 7.50-7.40 (m, 1H), 7.32-7.29 (m, 2H), 7.19-7.16 (m, 1H), 7.05 (d, 1H, J=2.5), 7.02 (dd, 1H, J=8.8, J=2.5), 6.35 (s, 1H), 5.23 (s, 2H), 3.86-3.80 (m, 2H), 2.73-2.69 (m, 2H), 1.20 (t, 3H, J=7.2).

### Example 15

### 4-[(Isopropylamino)methyl]-7-(3-fluorobenzyloxy)-2H-chromen-2-one

¹H-NMR (DMSO-d₆) δ: 7.78 (d, 1H, J=8.9), 7.51-7.41 (m, 1H), 7.33-7.30 (m, 2H), 7.20 (m, 1H), 7.06 (d, 1H, J=2.4), 7.02 (dd, 1H, J=8.9, J=2.4), 6.36 (s, 1H), 5.25 (s, 2H), 3.90-3.81 (m, 2H), 3.07 (m, 1H), 1.31 (d, 6H, J=6.5).

### Example 16

### 4-[(Dimethylamino)methyl]-7-(3-chlorobenzyloxy)-2H-chromen-2-one

Yield: 71%.

Mp: 78-80° C.

ESI-MS m/z: [MNa]⁺=366

¹H-NMR (CDCl₃) δ: 7.78 (d, 1H, J=8.8), 7.43 (s, 1H), 7.34-7.28 (m, 3H), 6.92 (dd, 1H, J=8.8, J=2.5), 6.86 (d, 1H, J=2.5), 6.33 (s, 1H), 5.10 (s, 2H), 3.53 (s, 2H), 2.33 (s, 6H).

### Example 17

### 4-[(Dimethylamino)methyl]-7-(3-fluorobenzyloxy)-2H-chromen-2-one

Yield: 74%.

Mp: 84-86° C.

ESI-MS m/z: [MNa]⁺=350.

¹H-NMR (CDCl₃) δ: 7.79 (d, 1H, J=8.8), 7.40-7.33 (m, 1H), 7.20-7.13 (m, 2H), 7.06-7.00 (m, 1H), 6.91 (dd, 1H, J=8.8, J=2.5), 6.85 (d, 1H, J=2.5), 6.31 (s, 1H), 5.12 (s, 2H), 3.51 (s, 2H), 2.32 (s, 6H).

### Example 18

### 4-[(Benzylamino)methyl]-7-(3-chlorobenzyloxy)-2H-chromen-2-one

A mixture of 402 mg (1.2 mmol) of 4-chloromethyl-7-(3-chlorobenzyloxy)-2H-chromen-2-one, 166 mg of K₂CO₃ (1.2 mmol) and 655 μL of benzylamine (6 mmol) was stirred in refluxing absolute ethanol (10 ml) for 5 hours. The reaction mixture was cooled to room temperature, the inorganic solid residue was filtered off, the solvent was evaporated and the resulting oil was purified by column chromatography on silica gel (eluant CHCl₃/n-hexane/AcOEt 7/2/1 v/v/v) giving a solid, which was crystallized from absolute ethanol yielding 137 mg (28%) of a yellow solid with a melting point of 133-135° C.

ESI-MS m/z: [MNa]⁺=428.

¹H-NMR (CDCl₃) δ: 7.54 (d, 1H, J=8.8), 7.43 (s, 1H), 7.39-7.27 (m, 8H), 6.90 (d, 1H, J=2.5), 6.87 (dd, 1H, J=8.8, J=2.5), 6.49 (s, 1H), 5.09 (s, 2H), 3.94 (s, 2H), 3.93 (s, 2H).

### Example 19

### 4-[[(N-Benzyl-N-methyl)amino]methyl]-7-(3-chlorobenzyloxy)-2H-chromen-2-one

This compound was prepared according to the same procedure of Example 18 by using N-benzyl-N-methylamine instead of benzylamine.

Yield: 46%.

Mp: 107-108° C.

ESI-MS m/z: [MNa]⁺=442.

¹H-NMR (DMSO-d₆) δ: 7.85 (d, 1H, J=8.8), 7.53 (s, 1H), 7.44-7.41 (m, 3H), 7.39-7.20 (m, 5H), 7.05 (d, 1H, J=1.9), 7.02 (dd, 1H, J=8.8, J=1.9), 6.35 (s, 1H), 5.23 (s, 2H), 3.67 (s, 2H), 3.58 (s, 2H), 2.13 (s, 3H).

### Example 20

### 4-[(Aminocarbonyl)methyl]-7-(3-chlorobenzyloxy)-2H-chromen-2-one

A mixture of 219 mg (1 mmol) of 4-[(aminocarbonyl)methyl]-7-hydroxy-2H-chromen-2-one, 0.353 ml (3 mmol) of 3-chlorobenzyl alcohol, 757 mg (3 mmol) of 1,1′-(azodicarbonyl)dipiperidine (ADDP) and 787 mg (3 mmol) of triphenylphosphine in 10 ml of anhydrous THF was stirred at room temperature for 18 hours. The precipitate was filtered off, and the solvent was evaporated under vacuum. The oily residue was treated with diethyl ether, obtaining a solid material which was crystallized from ethanol to give 158 mg (38%) of the title compound with a melting point of 185-186° C.

¹H-NMR (DMSO-d₆) δ: 7.68 (d, J=8.7, 1H); 7.63 (s, 1H); 7.54 (s, 1H); 7.44-7.38 (m, 3H); 7.17 (s, 1H); 7.08 (d, J=2.4, 1H); 7.05 (dd, J=8.8, J=2.4, 1H); 6.25 (s, 1H); 5.24 (s, 2H); 3.64 (s, 2H).

### Example 21

### 4-(2-Aminoethyl)-7-(3-chlorobenzyloxy)-2H-chromen-2-one

To a mixture of 33 mg (0.1 mmol) of 4-cyanomethyl-7-(3-chlorobenzyloxy)-2H-chromen-2-one and 48 mg (0.2 mmol) of CoCl₂.6H₂O in 2 ml of methanol, 38 mg (1 mmol) of sodium borohydride were added portionwise over 10 minutes. The suspension was stirred at room temperature for an additional hour and then 1 ml of 2 N HCl was added and the methanol stripped off under vacuum. The acidic solution was cooled to 0° C. and 5 ml of a 30% ammonia aqueous solution were added. The basic solution was extracted twice with ethyl acetate, the combined organic extracts were dried over anhydrous sodium sulphate, filtered and evaporated to dryness under vacuum to yield a yellow solid, which was dissolved in 2 ml of chloroform. Subsequently, 1 ml of 3 N HCl was added. After stirring, a white precipitate, corresponding to the hydrochloride salt of the title compound was obtained by filtration.

Yield: 30%.

Mp: 113° C. dec.

ESI-MS m/z, [MH]⁺=330.

¹H-NMR (DMSO-d₆) δ: 7.96 (b, 3H, exch. D₂O); 7.78 (d, J=8.8, 1H); 7.54 (s, 1H); 7.44-7.42 (m, 3H); 7.11 (d, J=2.5, 1H); 7.07 (dd, J=8.8, J=2.4, 1H); 6.27 (s, 1H); 5.26 (s, 2H); 3.08 (m, 4H).

### Example 22

### 4-[2-(Methylamino)ethyl]-7-(3-chlorobenzyloxy)-2H-chromen-2-one (NW-1801)

To 5.1 ml (10.2 mmol) of a 2.0 M solution of methylamine in THF, 200 mg (0.51 mmol) of 4-(2-bromoethyl)-7-(3-chlorobenzyloxy)-2H-chromen-2-one were added, followed by 70 mg (0.51 mmol) of anhydrous K₂CO₃ and 9 mg (0.051 mmol) of KI. The mixture was then stirred at 55° C. overnight. The precipitate was filtered off and the solvent was evaporated under vacuum to give an oily residue, which was purified by column chromatography on silica gel (eluant CHCl₃/MeOH 9:1 v/v) and crystallized from ethanol.

Yield: 29%.

Mp: 72° C. dec.

ESI-MS m/z, [MH]⁺=344.

¹H-NMR (DMSO-d₆) δ: 7.76 (d, J=8.8, 1H); 7.54 (s, 1H); 7.44-7.42 (m, 3H); 7.08 (d, J=2.5, 1H); 7.04 (dd, J=8.8, J=2.5, 1H); 6.19 (s, 1H); 5.24 (s, 2H); 2.92-2.84 (m, 4H); 2.34 (s, 3H).

The 4-aminomethyl coumarin derivatives synthesized could be easily transformed into their corresponding mesylate salts according to the following general procedure.

The 4-aminomethyl derivative (1.12 mmol) was dissolved in dry THF (6 ml) and methanesulfonic acid (80 μl, 1.23 mmol) was added. The formed solid salt was filtered and recrystallized from absolute ethanol.

Here below are reported, as an example, the physical characteristics of two of them.

### Example 23

### 4-[(Methylamino)methyl]-7-(3-chlorobenzyloxy)-2H-chromen-2-one methanesulfonate

Yield: 86%.

Mp: 213-215° C.

ESI/MS m/z: [MH]⁺=330.

¹H-NMR (DMSO-d₆) δ: 9.01 (s, 2H, exchange with D₂O), 7.77 (d, 1H, J=8.8), 7.54 (s, 1H), 7.44-7.37 (m, 3H), 7.14 (d, 1H, J=2.5), 7.10 (dd, 1H, J=8.8, J=2.5), 6.41 (s, 1H), 5.27 (s, 2H), 4.44 (s, 2H), 2.71 (s, 3H), 2.31 (s, 3H).

### Example 24

### 4-[(Methylamino)methyl]-7-(3-fluorobenzyloxy)-2H-chromen-2-one methanesulfonate

Yield: 90%.

Mp: 215-216° C.

ESI/MS m/z: [MH]⁺=314.

¹H-NMR (DMSO-d₆) δ: 8.96 (s, 2H, exchange with D₂O), 7.76 (d, 1H, J=8.8), 7.45-7.41 (m, 1H), 7.31-7.28 (m, 2H), 7.16 (m, 1H), 7.15 (d, 1H, J=2.5), 7.10 (dd, 1H, J=8.8, J=2.5), 6.40 (s, 1H), 5.27 (s, 2H), 4.43 (s, 2H), 2.70 (s, 3H), 2.28 (s, 3H).

If desired, a salt of a compound of formula (I) of this invention can be transformed in another salt or the corresponding free base by employing procedures commonly known in the art.

### Preparation of Intermediates

### A) 4-Chloromethyl-7-hydroxy-2H-chromen-2-one

Resorcinol (7.0 g, 63.6 mmol), ethyl 4-chloroacetoacetate (9.5 ml, 69.9 mmol) and 104 ml of 96% sulphuric acid were stirred for 2 hours at 0° C. The reaction mixture was poured into ice water (200 ml) and extracted with ethyl acetate. The organic layers were collected, washed with NaHCO₃ 10% aqueous solution, then with water, dried over sodium sulphate and evaporated under vacuum. The resulting oil was purified by column chromatography on silica gel (eluant CHCl₃/AcOEt 7.5/2.5 v/v) yielding 5.22 g (45.7%) of a white solid used without any further purification for the next step synthesis.

¹H-NMR (Acetone-d₆) δ: 9.50 (s, 1H, exchanges with D₂O), 7.73 (d, 1H, J=8.8), 6.91 (dd, 1H, J=8.8, J=2.5), 6.80 (d, 1H, J=2.5), 6.40 (s, 1H), 4.92 (s, 2H).

### B) 4-Chloromethyl-7-benzyloxy-2H-chromen-2-one

A mixture of 4-chloromethyl-7-hydroxy-2H-chromen-2-one (10.0 g, 47.5 mmol), anhydrous K₂CO₃ (6.56 g, 47.5 mmol) and benzyl bromide (12.2 g, 71.3 mmol) was stirred in refluxing absolute ethanol (300 ml) for 2 hours. The reaction mixture was cooled to room temperature and the inorganic precipitate filtered off. The solvent was evaporated under vacuum, the crude residue was treated with diethyl ether and filtered to give 9.86 g (yield 69.0%) of a white solid.

¹H-NMR (CDCl₃) δ: 7.57 (d, 1H, J=8.8), 7.45-7.34 (m, 5H), 6.97 (dd, 1H, J=8.8, J=2.5), 6.92 (d, 1H, J=2.5), 6.40 (s, 1H), 5.14 (s, 2H), 4.62 (s, 2H).

### C) 4-Chloromethyl-7-(3-chlorobenzyloxy)-2H-chromen-2-one

This compound was prepared according to the procedure of Example A) by using (3-chlorobenzyl)bromide instead of benzyl bromide.

Yield: 78%.

¹H-NMR (CDCl₃) δ: 7.58 (d, 1H, J=8.8), 7.43 (br, 1H), 7.37-7.27 (m, 3H), 6.96 (dd, 1H, J=8.8, J=2.5), 6.88 (d, 1H, J=2.5), 6.41 (s, 1H), 5.11 (s, 2H), 4.62 (s, 2H).

### D) 4-Chloromethyl-7-(3-fluorobenzyloxy)-2H-chromen-2-one

This compound was prepared according to the procedure of Example A) by using (3-fluorobenzyl)bromide instead of benzyl bromide.

Yield: 73%.

¹H-NMR (CDCl₃) δ: 7.58 (d, 1H, J=8.8), 7.41-7.34 (m, 1H), 7.25-7.13 (m, 2H), 7.07-7.01 (m, 1H), 6.97 (dd, 1H, J=8.8, J=2.5), 6.89 (d, 1H, J=2.5), 6.41 (s, 1H), 5.14 (s, 2H), 4.62 (s, 2H).

### E) 4-Azidomethyl-7-benzyloxy-2H-chromen-2-one

A mixture of 4-chloromethyl-7-benzyloxy-2H-chromen-2-one (511 mg, 1.7 mmol) and NaN₃ (442 mg, 6.8 mmol) was refluxed in absolute ethanol (17 ml) for 2 hours. The mixture was cooled to room temperature and the solid residue was filtered off. The solvent was evaporated under vacuum and the resulting oil was purified by column chromatography on silica gel (eluant n-hexane/AcOEt 8/2 v/v) yielding 460 mg (45%) of a yellow solid.

¹H-NMR (CDCl₃) δ: 7.46-7.34 (m, 6H), 6.97-6.96 (br, 1H), 6.93 (br, 1H), 6.36 (s, 1H), 5.14 (s, 2H), 4.51 (s, 2H).

### F) 4-Azidomethyl-7-(3-chlorobenzyloxy)-2H-chromen-2-one

This compound was prepared according to the procedure of Example E) by using 4-chloromethyl-7-(3-chlorobenzyloxy)-2H-chromen-2-one instead of 4-chloromethyl-7-benzyloxy-2H-chromen-2-one.

Yield: 47%.

¹H-NMR (CDCl₃) δ: 7.47-7.43 (m, 2H), 7.38-7.35 (m, 3H), 6.92 (dd, 1H, J=8.8, J=2.5), 6.88 (d, 1H, J=2.5), 6.38 (s, 1H), 5.09 (s, 2H), 4.50 (s, 2H).

### G) 4-Azidomethyl-7-(3-fluorobenzyloxy)-2H-chromen-2-one

This compound was prepared according to the procedure of Example E) by using 4-chloromethyl-7-(3-fluorobenzyloxy)-2H-chromen-2-one instead of 4-chloromethyl-7-benzyloxy-2H-chromen-2-one.

Yield: 43%.

¹H-NMR (CDCl₃) δ: 7.48-7.46 (d, 1H, J=8.8), 7.41-7.35 (m, 1H), 7.22-7.14 (m, 2H), 7.05-7.00 (m, 1H), 6.96 (dd, 1H, J=8.8, J=2.5), 6.92 (d, 1H, J=2.5), 6.39 (s, 1H), 5.10 (s, 2H), 4.52 (s, 2H).

### H) 4-[(Ethoxycarbonyl)methyl]-7-hydroxy-2H-chromen-2-one

Resorcinol (2.2 g, 20 mmol), diethyl-1,3-acetonedicarboxylate (4 ml, 22 mmol) and few drops of 96% sulphuric acid were stirred at 120° C. for 1 hour. The oily residue obtained was treated with ethanol yielding 1.99 g (40%) of a precipitate used without any further purification in the next synthetic step.

¹H-NMR (DMSO-d₆) δ: 10.55 (b, 1H); 7.49 (d, J=8.8, 1H); 6.78 (dd, J=8.8, J=2.3, 1H); 6.71 (d, J=2.3, 1H); 6.21 (s, 1H); 4.09 (q, J=7.1, 2H); 3.91 (s, 2H); 1.16 (t, J=7.1, 3H).

### I) 4-[(Ethoxycarbonyl)methyl]-7-(3-chlorobenzyloxy)-2H-chromen-2-one

A solution of 0.124 g (0.5 mmol) of 4-[(ethoxycarbonyl)methyl]-7-hydroxy-2H-chromen-2-one, 0.177 ml (1.5 mmol) of 3-chlorobenzyl alcohol, 0.378 g (1.50 mmol) of 1,1′-(azodicarbonyl)dipiperidine (ADDP) and 0.393 g (1.5 mmol) of triphenylphosphine in 5 ml of anhydrous THF was stirred at room temperature for 18 hours. The precipitate was filtered off, the solvent evaporated under vacuum and the oily residue purified by flash chromatography on silica gel (eluant CHCl₃).

Yield: 48%.

GC-MS (EI) M⁺372.

¹H NMR (DMSO-d₆) δ: 7.67 (d, J=8.8, 1H); 7.53 (s, 1H); 7.42-7.39 (m, 3H); 7.08-7.01 (m, 2H); 6.23 (s, 1H); 5.23 (s, 2H); 4.09 (q, J=7.1, 2H); 3.91 (s, 2H); 1.16 (t, J=7.1, 3H).

### J) 4-[(Aminocarbonyl)methyl]-7-hydroxy-2H-chromen-2-one

A sealed glass ampoule containing 800 mg (3.23 mmol) of 4-[(ethoxycarbonyl)methyl]-7-hydroxy-2H-chromen-2-one and 8 ml (16 mmol) of a 2.0 M solution of ammonia in methanol was placed in an oven at 90° C. for 60 hours. The solution was then evaporated to dryness under vacuum and the residue was crystallized from ethanol to give 354 mg (50%) of a white solid.

¹H-NMR (DMSO-d₆) δ: 7.68 (d, J=8.7, 1H); 7.63 (s, 1H); 7.17 (s, 1H); 7.08 (d, J=2.4, 1H); 7.05 (dd, J=8.8, J=2.4, 1H); 6.25 (s, 1H); 3.64 (s, 2H).

### K) 4-[(Dimethylaminocarbonyl)methyl]-7-hydroxy-2H-chromen-2-one

This compound was prepared according to the procedure of Example J) by using dimethylamine instead of ammonia.

¹H-NMR (DMSO-d₆) δ: 7.46 (d, J=8.8, 1H); 6.75 (dd, J=8.8, J=2.2, 1H); 6.69 (d, J=2.2, 1H); 6.06 (s, 1H); 3.89 (s, 2H); 3.06 (s, 3H); 2.83 (s, 3H).

### L) 4-[(Dimethylaminocarbonyl)methyl]-7-benzyloxy-2H-chromen-2-one

To a solution of 0.05 g (0.2 mmol) of 4-[(dimethylaminocarbonyl)methyl]-7-hydroxy-2H-chromen-2-one in absolute ethanol, 0.055 g of K₂CO₃ (0.4 mmol) and 0.071 ml of benzyl bromide (0.6 mmol) were added. The mixture was refluxed for 30 minutes. The precipitate was filtered off from the hot solution, that was then cooled to room temperature. The crystalline precipitate formed was collected by filtration.

Yield: 55%.

Mp: 162-163° C.

¹H-NMR (DMSO-d₆) δ: 7.55 (d, J=8.8, 1H); 7.47-7.30 (m, 5H); 7.06 (d, J=2.5, 1H); 6.99 (dd, J=8.8, J=2.5, 1H); 6.15 (s, 1H); 5.21 (s, 2H); 3.93 (s, 2H); 3.07 (s, 3H); 2.83 (s, 3H).

### M) 4-[(tert-Butoxycarbonylhydrazinocarbonyl)methyl]-7-hydroxy-2H-chromen-2-one

A solution of 0.44 g (2 mmol) of 7-hydroxycoumarin-4-acetic acid, 0.92 g (6 mmol) of hydroxybenzotriazole, 1.24 g (6 mmol) of dicyclohexylcarbodiimide and 0.79 g (6 mmol) of tert-butyl carbazate in 12 ml of anhydrous DMF, was stirred at room temperature for 5 hours. The precipitate was filtered off and the solvent was evaporated under vacuum yielding a solid residue that was treated with chloroform to give the title compound (98% yield), used without any further purification for the next synthesis.

¹H-NMR (DMSO-d₆) δ: 10.57 (s, 1H); 9.93 (s, 1H); 8.85 (s, 1H); 7.61 (d, J=8.7, 1H); 6.77 (dd, J=8.7, J=2.4, 1H); 6.70 (d, J=2.4, 1H); 6.22 (s, 1H); 3.60 (s, 2H); 1.40 (s, 9H).

### N) 4-[(tert-Butoxycarbonylhydrazinocarbonyl)methyl]-7-benzyloxy-2H-chromen-2-one

Benzyl bromide, 0.18 ml (1.5 mmol), was added to a mixture of 0.5 g (1.5 mmol) of 4[(tert-butoxycarbonylhydrazinocarbonyl)methyl]-7-hydroxy-2H-chromen-2-one and 0.21 g (1.5 mmol) of K₂CO₃ in absolute ethanol. The resulting mixture was refluxed for 30 minutes. The solid was filtered off and the solution was cooled to room temperature. The solvent was evaporated under vacuum to. The resulting solid was purified by column chromatography on silica gel (eluant CHCl₃/MeOH 9.5/0.5 v/v) to give the title compound in 30% yield.

¹H-NMR (DMSO-d₆) δ: 9.94 (s, 1H); 8.85 (s, 1H); 7.70 (d, J=8.8, 1H); 7.46-7.30 (m, 5H); 7.08 (d, J=2.2, 1H); 7.01 (dd, J=8.8, J=2.2, 1H); 6.30 (s, 1H); 5.22 (s, 2H); 3.68 (s, 2H); 1.37 (s, 9H).

### O) 4-[[(2-tert-Butoxycarbonylaminoethyl)aminocarbonyl]methyl]-7-hydroxy-2H-chromen-2-one

To a solution of 0.22 g (1 mmol) of 7-hydroxycoumarin-4-acetic acid and 0.41 g (2 mmol) of dicyclohexylcarbodiimide in 6 ml of anhydrous DMF, 0.27 g (2 mmol) of hydroxybenzotriazole and 0.32 g (2 mmol) of N-Boc-ethylenediamine were added. The mixture was stirred at room temperature for 5 hours. The precipitate was filtered off and the solvent was evaporated under vacuum. The residue was crystallized from CHCl₃/n-hexane to give the title compound in 65% yield.

¹H-NMR (DMSO-d₆) δ: 10.54 (b, 1H); 8.19 (b, 1H); 7.57 (d, J=8.8, 1H); 6.78-6.69 (m, 3H); 6.14 (s, 1H); 3.60 (s, 2H); 3.05-2.96 (m, 4H); 1.35 (s, 9H).

### P) 4-[[(2-tert-Butoxycarbonylaminoethyl)aminocarbonyl]methyl]-7-(3-chlorobenzyloxy)-2H-chromen-2-one

A solution of 0.23 g (0.64 mmol) of 4-[[(2-tert-butoxyaminoethyl)aminocarbonyl]methyl]-7-hydroxy-2H-chromen-2-one, 0.224 ml (1.9 mmol) of 3-chlorobenzyl alcohol, 0.48 g (1.9 mmol) of 1,1′-azodicarbonyl-dipiperidine (ADDP) and 0.5 g (1.9 mmol) of triphenyl-phosphine in 7 ml of anhydrous THF was stirred at room temperature for 18 hours. The precipitate was filtered off, the solvent was evaporated under vacuum and the oily residue was treated with diethyl ether to give a solid (95% yield) which was used without any further purification for the preparation of the compound of Example 11.

¹H-NMR (DMSO-d₆) δ: 8.21 (b, 1H); 7.67 (d, J=8.8, 1H); 7.53 (s, 1H); 7.41-7.39 (m, 3H); 7.07 (d, J=2.5, 1H); 7.03 (dd, J=8.8, J=2.5, 1H); 6.81 (b, 1H); 6.23 (s, 1H); 5.23 (s, 2H); 3.64 (s, 2H); 3.07-3.03 (m, 2H); 2.98-2.94 (m, 2H); 1.35 (s, 9H).

### Q) 4-Cyanomethyl-7-(3-chlorobenzyloxy)-2H-chromen-2-one

To a solution of 125 mg (0.38 mmol) of 4-[(aminocarbonyl)methyl]-7-(3-chlorobenzyloxy)-2H-chromen-2-one and 0.061 ml (0.76 mmol) of anhydrous pyridine in 4 ml of anhydrous dioxane, 0.068 ml (0.48 mmol) of trifluoracetic anhydride were added dropwise at 0° C. The clear solution was allowed to reach room temperature and was poured into ice. The aqueous solution was extracted twice with chloroform, the combined organic phases were dried over anhydrous sodium sulphate, filtered and evaporated to dryness under vacuum to give after crystallization from ethanol 120 mg (97%) of a white solid.

¹H-NMR (DMSO-d₆), δ: 7.67 (d, J=8.8, 1H); 7.54 (s, 1H); 7.44-7.37 (m, 3H); 7.14-7.12 (m, 1H); 7.09 (d, J=2.5, 1H); 6.33 (s, 1H); 5.25 (s, 2H); 4.37 (s, 2H).

### R) 4-(2-Hydroxyethyl)-7-hydroxy-2H-chromen-2-one

To a solution of 937 mg (4.26 mmol) of 7-hydroxycoumarin-4-acetic acid in 25 ml of anhydrous THF, 12.8 ml of a 1.0 M solution of borane in THF were added dropwise at 0° C. The mixture was allowed to reach room temperature and was stirred for 6 additional hours. The reaction mixture was cooled to 0° C. and then 20 ml of methanol were added. The solvent was evaporated under vacuum and the residue was dissolved in ethyl acetate, washed with water, dried over anhydrous sodium sulphate, filtered and evaporated to dryness to give a solid residue. After purification by flash chromatography on silica gel (eluant CHCl₃/MeOH 9:1 v/v) 474 mg (54%) of a white solid were obtained.

¹H-NMR (DMSO-d₆) δ: 10.54 (s, 1H); 7.63 (d, J=8.7, 1H); 6.78 (dd, J=8.7, J=2.2, 1H); 6.70 (d, J=2.2, 1H); 6.09 (s, 1H); 4.80 (t, J=5.2, 1H); 3.71-3.65 (m, 2H); 2.86 (t, J=6.3, 2H).

### S) 4-(2-Hydroxyethyl)-7-benzyloxy-2H-chromen-2-one

To a mixture of 206 mg (1 mmol) of 4-(2-hydroxyethyl)-7-hydroxy-2H-chromen-2-one and 138 mg of K₂CO₃ (1 mmol) in 5 ml of absolute ethanol, 342 mg (2 mmol) of benzyl bromide were added and the mixture was refluxed for 45 minutes. The solid material was filtered off and the organic solution was evaporated to dryness under vacuum. The oily residue was purified by flash chromatography on silica gel (eluant CHCl₃/MeOH 9.5:0.5 v/v) to give 157 mg (53%) of a white solid, used without any further purification in the subsequent synthetic step.

¹H-NMR (DMSO-d₆) δ: 7.73 (d, J=8.8, 1H); 7.47-7.30 (m, 5H); 7.06 (d, J=2.5, 1H); 7.01 (dd, J=8.8, J=2.5, 1H); 6.17 (s, 1H); 5.21 (s, 2H); 4.80 (t, J=5.5, 1H); 3.72-3.66 (m, 2H); 2.89 (t, J=6.3, 2H).

### T) 4-(2-Hydroxyethyl)-7-(3-chlorobenzyloxy)-2H-chromen-2-one

This compound was prepared according to the same procedure of Example S) by using (3-chlorobenzyl)bromide instead of benzyl bromide.

Yield: 86%.

¹H-NMR (DMSO-d₆) δ: 7.74 (d, J=8.6, 1H); 7.53 (s, 1H); 7.43-7.41 (m, 3H); 7.06 (d, J=2.5, 1H); 7.02 (dd, J=8.6, J=2.5, 1H); 6.18 (s, 1H); 5.23 (s, 2H); 4.78 (b, 1H); 3.69 (t, J=6.3, 2H); 2.89 (t, J=6.3, 2H).

### U) 4-(2-Bromoethyl)-7-benzyloxy-2H-chromen-2-one

To a solution of 296 mg (1 mmol) of 4-(2-hydroxyethyl)-7-benzyloxy-2H-chromen-2-one and 730 mg (2.2 mmol) of carbon tetrabromide in 10 ml of anhydrous dichloromethane, 525 mg (2 mmol) of triphenylphosphine, dissolved in 2 ml of anhydrous dichloromethane were added dropwise at 0° C. The mixture was allowed to reach room temperature and stirred for additional 60 minutes. The solvent was evaporated under vacuum and the resulting oily residue was purified by flash chromatography on silica gel (eluant CHCl₃/n-hexane 8:2 v/v) to give 295 mg (82%) of a white solid.

¹H-NMR (DMSO-d₆) δ: 7.75 (d, J=9.0, 1H); 7.47-7.30 (m, 5H); 7.08 (d, J=2.2, 1H); 7.02 (dd, J=9.0, J=2.2, 1H); 6.27 (s, 1H); 5.21 (s, 2H); 3.82 (t, J=6.8, 2H); 3.34 (t, J=6.8, 2H).

### V) 4-(2-Bromoethyl)-7-(3-chlorobenzyloxy)-2H-chromen-2-one

This compound was prepared according to the same procedure of Example U) by using 4-(2-hydroxyethyl)-7-(3-chloromethyloxy)-2H-chromen-2-one instead of 4-(2-hydroxyethyl)-7-benzyloxy-2H-chromen-2-one.

Yield: 59%.

¹H-NMR (CDCl₃) δ: 7.50 (d, J=8.8, 1H); 7.44 (s, 1H); 7.33-7.32 (m, 3H); 6.95 (dd, J=8.8, J=2.5, 1H); 6.89 (d, J=2.5, 1H); 6.20 (s, 1H); 5.11 (s, 2H); 3.64 (t, J=7.2, 2H); 3.30 (t, J=7.2, 2H).

### Experimental Pharmacology

### In Vitro MAO-A and MAO-B Enzyme Activities Assay

Membrane Preparations (Crude Mitochondrial Fraction)

Male Wistar rats (Harlan, Italy—175-200 g) were sacrificed under light anaesthesia and brains were rapidly removed and homogenized in 8 volumes of ice-cold 0.32 M sucrose buffer containing 0.1 M EDTA, pH 7.4. The crude homogenate was centrifuged at 2220 rpm for 10 minutes at +4° C. and the supernatant recovered. The pellet was homogenized and centrifuged again. The two supernatants were pooled and centrifuged at 9250 rpm for 10 minutes. The pellet was resuspended in fresh buffer and centrifuged at 11250 rpm for 10 minutes at +4° C. The resulting pellet was stored at −80° C.

In Vitro Enzyme Activities Assay

The enzyme activities were assessed with a radioenzymatic assay using the substrates ¹⁴C-serotonin (5-HT) and ¹⁴C-phenylethylamine (PEA) for MAO-A and MAO-B, respectively.

The mitochondrial pellet (500 μg protein) was resuspended in 0.1 M phosphate buffer (pH 7.4). 500 μl of the suspension were added to a 50 μl solution of the test compound or buffer, and incubated for 30 min at 37° C. (preincubation) then the substrate (50 μl) was added. The incubation was carried out for 30 minutes at 37° C. (¹⁴C-5-HT, 5 μM) or for 10 minutes at 37° C. (¹⁴C-PEA, 0.5 μM).

The reaction was stopped by adding 0.2 ml of 37% HCl or perchloric acid. After centrifugation, the deaminated metabolites were extracted with 3 ml of diethyl ether (5-HT) or toluene (PEA) and the radioactive organic phase was measured by liquid scintillation spectrometry at 90% efficiency. The amount of neutral and/or acidic metabolites formed as a result of MAO activity was obtained by measuring the radioactivity of the eluate.

The activity of MAO in the sample, corresponding to a percentage of radioactivity compared with the control activity in the absence of the inhibitor, was expressed as nmoles of substrate transformed/mg protein/min.

The drug inhibition curves were obtained from at least eight different concentration points, each in duplicate (10⁻¹⁰ to 10⁻⁵ M). The IC₅₀ values (the drug concentration inhibiting 50% of the enzyme activity) were calculated with confidence intervals determined using non linear regression analysis (best fitting aided-computer program). The compounds of this invention are able to selectively inhibit MAO-B in vitro, with a potency (IC₅₀) in the nanomolar range and with generally no relevant effect on MAO-A, as shown in Table 1.

TABLE 1

| | IC₅₀ [μM] | |
|---|---|---|
| COMPOUNDS | MAO-A | MAO-B |
| 4-[(Hydrazinocarbonyl)methyl]-7- | 1.4 | 0.04 |
| benzyloxy-2H-chromen-2-one | | |
| 4-[(Aminocarbonyl)methyl]-7-(3-fluoro | 70.0 | 0.05 |
| benzyloxy)-2H-chromen-2-one | | |
| 4-[(Dimethylaminocarbonyl)methyl]-7-(3-chloro | >100 | 0.04 |
| benzyloxy)-2H-chromen-2-one | | |
| 4-[(Dimethylaminocarbonyl)methyl]-7- | 15.3 | 0.08 |
| benzyloxy-2H-chromene | | |
| 4-Aminomethyl-7-(3-chlorobenzyloxy)- | 1.9 | 0.01 |
| 2H-chromen-2-one | | |
| 4-[(Methylamino)methyl]-7-(3-fluorobenzyloxy)- | 13.5 | 0.02 |
| 2H-chromen-2-one | | |
| 4-(2-Aminoethyl)-7-(3-chlorobenzyloxy)- | 31.8 | 0.25 |
| 2H-chromen-2-one | | |
| 4-[(Methylamino)methyl]-7-(3-chlorobenzyloxy)- | 5.9 | 0.01 |
| 2H-chromen-2-one | | |
| 4-[2-(Methylamino)ethyl]-7-(3-chlorobenzyloxy)- | 74.0 | 0.30 |
| 2H-chromen-2-one | | |

### In Vitro MAO-B Inhibition Reversibility Studies

To investigate whether the test compound was an irreversible or a reversible MAO-B inhibitor, the inhibition of the enzymatic activity was assessed using the following experimental protocols:

Time-Dependent Experiments:

the time-dependent association kinetics were deduced from the IC₅₀ values obtained without and with 30 minutes enzyme-inhibitor pre-incubation. For mechanism-based irreversible inhibitors that act by blocking the enzyme catalytic site, the inhibitory potency increases with incubation time. The absence of significant difference between IC₅₀ obtained from one or the other protocol is indicative of reversible inhibitors.

### Ex Vivo MAO-B Inhibition

Test compounds were administered orally to male C57BL mice (Harlan, Italy, 25-27 g) at the single dose of 10 mg/Kg. At various time intervals (1, 2, 4, 8 and 24 h), animals were sacrificed, brains removed, cortices dissected out and stored at −80° C. Crude homogenates (0.5%) were prepared in 0.1 M phosphate buffer (pH 7.4) and were freshly used. MAO-A and MAO-B activity were assessed as described above. After oral, single dose administration in mice, the compounds of this invention behave as potent and reversible, short-acting MAO-B inhibitors with full recovery of the MAO-B enzymatic activity 8-16 hours after the administration.

1. A compound of formula (I) 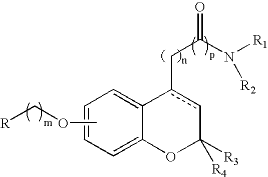
wherein:
the group 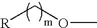
is a substituent in position 6 or 7 wherein:
R is a mono- or bi-cyclic (C₆-C₁₀) aryl radical or a mono- or bi-cyclic (5-10) membered heteroaryl radical, said radicals being optionally substituted by one or two substituents selected from (C₁-C₅) straight or branched alkyl, (C₁-C₅) straight or branched alkoxy, hydroxy, halo and trifluoromethyl;
m is zero or an integer from 1 to 3;
R₁ and R₂ each independently represent:
hydrogen;
(C₁-C₅) straight or branched alkyl optionally substituted by phenyl, where the phenyl group is optionally substituted by one or two substituents selected from (C₁-C₅) straight or branched alkyl, hydroxy, (C₁-C₅) straight or branched alkoxy, halo and trifluoromethyl;
(C₂-C₅) straight or branched alkyl substituted by amino;
phenyl, where the phenyl group is optionally substituted by one or two substituents selected from (C₁-C₅) straight or branched alkyl, hydroxy, (C₁-C₅) straight or branched alkoxy, halo and trifluoromethyl;
amino, (C₁-C₅) straight or branched alkyl- or dialkyl-amino;
or R₁ and R₂, taken together with the adjacent nitrogen atom form a saturated 5 to 7 member heterocyclic ring optionally containing one or two additional heteroatoms or groups selected from O, S and NR₅, wherein R₅ is hydrogen or a (C₁-C₅) straight or branched alkyl;
n is an integer from 1 to 3;
p is zero or 1;
R₃ and R₄ are both hydrogen, or taken together represent an oxygen atom;
the dotted line indicates nil or an additional bond;
with the proviso that:
(i) when R, m, n, p, R₃, R₄ and the dotted line are as above and one of R₁ and R₂ represents amino or (C₁-C₅) straight or branched alkylamino, then the other represents hydrogen or (C₁-C₅) straight or branched alkyl group;
(ii) when m and the dotted line are as above, n is 1, p is zero, R is a mono- or bi-cyclic (C₆-C₁₀) aryl radical optionally substituted as indicated above, R₃ and R₄ are both hydrogen, and one of R₁ and R₂ is hydrogen or (C₁-C₅) straight or branched alkyl, then the other may not be a (C₂-C₅) straight or branched alkyl substituted with phenyl where the phenyl group may be optionally substituted by one or two substituents as defined above;
(iii) when m is an integer from 1 to 3, n, p are as defined above, the dotted line indicates an additional bond; and
R₁ and R₂ each independently represent:
hydrogen;
(C₁-C₅) straight or branched alkyl optionally substituted by phenyl, where the phenyl group is optionally substituted by one or two substituents selected from (C₁-C₅) straight or branched alkyl, hydroxy, (C₁-C₅) straight or branched alkoxy, halo and trifluoromethyl;
(C₂-C₅) straight or branched alkyl substituted by amino; phenyl, where the phenyl group is optionally substituted by one or two substituents selected from (C₁-C₅) straight or branched alkyl, hydroxy, (C₁-C₅) straight or branched alkoxy, halo and trifluoromethyl;
or, when p is zero, R₁ and R₂, taken together with the adjacent nitrogen atom form a saturated 5 to 6 member heterocyclic ring optionally containing one additional heteroatom or group selected from O, S and NR₅, wherein R₅ is hydrogen or a (C₁-C₅) straight or branched alkyl; and
R₃ and R₄ taken together represent an oxygen atom; and 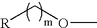
is a substituent in position 7;
then R cannot represent an unsubstituted mono- or bi-cyclic (C₆-C₁₀) aryl radical;
if the case, either as single optical isomers or mixtures thereof, and the pharmaceutically acceptable salts thereof.

2. A compound of claim 1 wherein:
R is phenyl substituted by one or two substituents selected from (C₁-C₄) straight or branched alkyl, (C₁-C₄) straight or branched alkoxy, halo, and trifluoromethyl, or R is pyridyl;
m is zero, 1 or 2;
R₁ and R₂ each independently represents hydrogen, (C₁-C₄) straight or branched alkyl or phenyl-(C₁-C₂) alkyl; or one of R₁ and R₂ represents amino and the other represents hydrogen or (C₁-C₄) straight or branched alkyl; or R₁ and R₂ taken together with the adjacent nitrogen atom form a saturated 5 to 6 membered heterocyclic ring optionally containing an additional heteroatom selected from O, S and N(C₁-C₄) straight or branched alkyl;
n is 1, 2 or 3;
p is zero or 1;
R₃ and R₄ taken together represent an oxygen atom;
the dotted line indicates nil or an additional bond;
if the case, as a single optical isomer or a mixture thereof, and the pharmaceutically acceptable salts thereof.

3. A compound of claim 1 wherein
R is phenyl substituted by one substituent selected from (C₁-C₃) straight or branched alkyl, (C₁-C₃) straight or branched alkoxy, fluoro, chloro, and trifluoromethyl, or R is pyridyl;
m is 1;
R₁ and R₂ each independently represent hydrogen, (C₁-C₃) straight or branched alkyl or benzyl; or one of R₁ and R₂ represents amino and the other represents hydrogen or (C₁-C₃) straight or branched alkyl; or R₁ and R₂ taken together with the adjacent nitrogen atom form a saturated 5 to 6 membered heterocyclic ring containing one additional heteroatom selected from O, S and N(C₁-C₃) straight or branched alkyl;
n is 1 or 2;
p is zero or 1;
R₃ and R₄ taken together represents an oxygen atom;
the dotted line indicates an additional bond;
if the case, as a single optical isomer or a mixture thereof, and the pharmaceutically acceptable salts thereof.

4. A compound of claim 1 wherein:
R is phenyl optionally substituted by one or two substituents selected from (C₁-C₄) straight or branched alkyl, (C₁-C₄) straight or branched alkoxy, halo and trifluoromethyl, or R is pyridyl;
m is zero, 1 or 2;
R₁ and R₂ each independently represents hydrogen, (C₁-C₄) straight or branched alkyl or benzyl; or one of R₁ and R₂ represents amino and the other represents hydrogen or (C₁-C₄) straight or branched alkyl; or R₁ and R₂ taken together with the adjacent nitrogen atom form a saturated 5 to 6 membered heterocyclic ring optionally containing an additional heteroatom selected from O, S, and N(C₁-C₃) straight or branched alkyl;
n is 1, 2 or 3;
p is zero or 1;
R₃ and R₄ are both hydrogen;
the dotted line indicates nil or an additional bond;
if the case, as a single optical isomer or a mixture thereof, and the pharmaceutically acceptable salts thereof.

5. A compound of claim 1 wherein:
R is phenyl optionally substituted by one substituent selected from (C₁-C₃) straight or branched alkyl, (C₁-C₃) straight or branched alkoxy, fluoro, chloro and trifluoromethyl, or R is pyridyl;
m is 1;
R₁ and R₂ each independently represent hydrogen or (C₁-C₃) straight or branched alkyl or benzyl; or one of R₁ and R₂ represent amino and the other represents hydrogen or (C₁-C₃) straight or branched alkyl; or R₁ and R₂ taken together with the adjacent nitrogen atom form a saturated 5 to 6 membered heterocyclic ring containing one additional heteroatom selected from O, S and N(C₁-C₃) straight or branched alkyl;
n is 1 or 2;
p is zero or 1;
R₃ and R₄ are both hydrogen;
the dotted line indicates nil or an additional bond;
if the case, as a single optical isomer or a mixture thereof, and the pharmaceutically acceptable salts thereof.

6. A compound of claim 1, selected from
4-[(Hydrazinocarbonyl)methyl]-7-benzyloxy-2H-chromen-2-one;
4-[(Aminocarbonypmethyl]-7-(3-hydroxybenzyloxy)-2H-chromen-2-one;
4-[(Aminocarbonyl)methyl]-7-(pyridin-3-yl)methoxy-2H-chromen-2-one;
4-[(Aminocarbonyl)methyl]-7-(pyridin-4-yl)methoxy-2H-chromen-2-one;
4-[(Aminocarbonypmethyl]-7-(3-chlorobenzyloxy)-2H-chromen-2-one;
4-[(Hydrazinocarbonyl)methyl]-6-(3-chlorobenzyloxy)-2H-chromen-2-one;
4-[(Hydrazinocarbonyl)methyl]-7-(3-chlorobenzyloxy)-2H-chromen-2-one;
4-[(Methylaminocarbonyl)methyl]-7-(3-chlorobenzyloxy)-2H-chromen-2-one;
4-[(Butylaminocarbonyl)methyl]-7-(3-chlorobenzyloxy)-2H-chromen-2-one;
4-[(Benzylaminocarbonyl)methyl]-7-(3-chlorobenzyloxy)-2H-chromen-2-one;
4-[(Dimethylaminocarbonyl)methyl]-7-(3-chlorobenzyloxy)-2H-chromen-2-one;
4-[(N-Butyl-N-methylaminocarbonyl)methyl]-7-(3-chlorobenzyloxy)-2H-chromen-2-one;
4-[[(2-Aminoethyl)aminocarbonyl]methyl]-7-(3-chlorobenzyloxy)-2H-chromen-2-one;
4-[(Aminocarbonypmethyl]-6-(3-fluorobenzyloxy)-2H-chromen-2-one;
4-[(Aminocarbonyl)methyl]-7-(3-fluorobenzyloxy)-2H-chromen-2-one;
4-[(Hydrazinocarbonyl)methyl]-7-(3-fluorobenzyloxy)-2H-chromen-2-one;
4-[(Methylaminocarbonyl)methyl]-7-(3-fluorobenzyloxy)-2H-chromen-2-one;
4-[(Benzylaminocarbony)methyl]-7-(3-fluorobenzyloxy)-2H-chromen-2-one;
4-[(Dimethylaminocarbonyl)methyl]-7-(3-fluorobenzyloxy)-2H-chromen-2-one;
4[2-(Hydrazinocarbonyl)ethyl]-7-benzyloxy-2H-chromen-2-one;
4-[2-(Aminocarbonyl)methyl]-7-(3-chlorobenzyloxy)-2H-chromen-2-one;
4-[2-(Hydrazinocarbonyl)ethyl]-7-(3-chlorobenzyloxy)-2H-chromen-2-one;
4-[2-(Methylaminocarbonyl)ethyl]-7-(3-chlorobenzyloxy)-2H-chromen-2-one;
4-[2-(Benzylaminocarbonyl)ethyl]-6-(3-chlorobenzyloxy)-2H-chromen-2-one;
4-[2-(Benzylaminocarbonyl)ethyl]-7-(3-chlorobenzyloxy)-2H-chromen-2-one;
4-[2-(Dimethylaminocarbonyl)ethyl]-7-(3-chlorobenzyloxy)-2H-chromen-2-one;
4-[2-(Aminocarbonyl)ethyl]-7-(3-fluorobenzyloxy)-2H-chromen-2-one;
4-[2-(Hydrazinocarbonyl)ethyl]-7-(3-fluorobenzyloxy)-2H-chromen-2-one;
4-[2-(Methylaminocarbonyl)ethyl]-7-(3-fluorobenzyloxy)-2H-chromen-2-one;
4-[2-(Butylaminocarbonyl)ethyl]-7-(3-fluorobenzyloxy)-2H-chromen-2-one;
4-[2-(Benzylaminocarbonyl)ethyl]-7-(3-fluorobenzyloxy)-2H-chromen-2-one;
4-[2-(Dimethylaminocarbonyl)ethyl]-7-(3-fluorobenzyloxy)-2H-chromen-2-one;
4-[(Aminocarbonyl)methyl]-7-benzyloxy-2H-chromene;
4-[(Hydrazinocarbonyl)methyl]-7-benzyloxy-2H-chromene;
4-[(Methylaminocarbonyl)methyl]-7-benzyloxy-2H-chromene;
4-[(Dimethylaminocarbonyl)methyl]-7-benzyloxy-2H-chromene;
4-[(Dimethylaminocarbonyl)methyl]-7-(3-chlorobenzyloxy)-2H-chromene;
4-[(Aminocarbonyl)methyl]-7-(3-fluorobenzyloxy)-2H-chromene;
4-[(Hydrazinocarbonyl)methyl]-7-(3-fluorobenzyloxy)-2H-chromene;
4-[(Methylaminocarbonyl)methyl]-7-(3-fluorobenzyloxy)-2H-chromene;
4-[(Dimethylaminocarbonyl)methyl]-7-(3-fluorobenzyloxy)-2H-chromene;
4-[2-(Aminocarbonyl)ethyl]-6-benzyloxy-2H-chromene;
4[2-(Aminocarbonyl)ethyl]-7-benzyloxy-2H-chromene;
4[2-(Hydrazinocarbonyl)ethyl]-7-benzyloxy-2H-chromene;
4-[2-(Methylaminocarbonyl)ethyl]-7-benzyloxy-2H-chromene;
4[2-(Dimethylaminocarbonyl)ethyl]-7-benzyloxy-2H-chromene;
4-[2-(Aminocarbonyl)ethyl]-7-(3-fluorobenzyloxy)-2H-chromene;
4[2-(Hydrazinocarbonyl)ethyl]-7-(3-fluorobenzyloxy)-211-chromene;
4-[2-(Methylaminocarbonyl)ethyl]-7-(3-fluorobenzyloxy)-2H-chromene;
4-[(Aminocarbonypmethyl]-7-benzyloxy-chroman;
4-[(Hydrazinocarbonyl)methyl]-7-benzyloxy-chroman;
4-[(Methylaminocarbonyl)methyl]-7-benzyloxy-chroman;
4-[(Aminocarbonypmethyl]-7-(3-fluorobenzyloxy)-chroman;
4-[(Hydrazinocarbonyl)methyl]-7-(3-fluorobenzyloxy)-chroman;
4-[(Methylaminocarbonyl)methyl]-7-(3-fluorobenzyloxy)-chroman;
4[2-(Hydrazinocarbonyl)ethyl]-7-benzyloxy-chroman;
4[2-(Methylaminocarbonyl)ethyl]-7-benzyloxy-chroman;
4[2-(Aminocarbonyl)ethyl]-7-(3-fluorobenzyloxy)-chroman;
4[2-(Methylaminocarbonyl)ethyl]-7-(3-fluorobenzyloxy)-chroman;
4-Aminomethyl-7-(3-chlorobenzyloxy)-2H-chromen-2-one;
4-(2-Aminoethyl)-7-(3-chlorobenzyloxy)-2H-chromen-2-one;
4-[(Methylamino)methyl]-7-(3-chlorobenzyloxy)-2H-chromen-2-one;
4-[(Dimethylamino)methyl]-6-(3-chlorobenzyloxy)-2H-chromen-2-one;
4-[(Dimethylamino)methyl]-7-(3-chlorobenzyloxy)-2H-chromen-2-one;
4-[2-(Methylamino)ethyl]-7-(3-chlorobenzyloxy)-2H-chromen-2-one;
4-[(Ethylamino)methyl]-7-(3-chlorobenzyloxy)-2H-chromen-2-one;
4-[2-(Ethylamino)ethyl]-7-(3-chlorobenzyloxy)-2H-chromen-2-one;
4-[(Benzylamino)methyl]-7-(3-chlorobenzyloxy)-2H-chromen-2-one;
4-[(N-Benzyl-N-methylamino)methyl]-7-(3-chlorobenzyloxy)-2H-chromen-2-one;
4-Aminomethyl-7-(3-fluorobenzyloxy)-2H-chromen-2-one;
4-[(Methylamino)methyl]-7-(3-fluorobenzyloxy)-2H-chromen-2-one;
4-[(Dimethylamino)methyl]-7-(3-fluorobenzyloxy)-2H-chromen-2-one;
4-[2-(Methylamino)ethyl]-7-(3-fluorobenzyloxy)-2H-chromen-2-one;
4-[(Ethylamino)methyl]-7-(3-fluorobenzyloxy)-2H-chromen-2-one;
4-[(Isopropylamino)methyl]-7-(3-fluorobenzyloxy)-2H-chromen-2-one;
4-(2-Aminoethyl)-7-benzyloxy-2H-chromene;
4-[2-(Methylamino)ethyl]-7-benzyloxy-2H-chromene;
4-(2-Aminoethyl)-7-(3-chlorobenzyloxy)-2H-chromene;
4-(2-Aminoethyl)-7-(3-fluorobenzyloxy)-2H-chromene;
4-(2-Aminoethyl)-6-benzyloxy-chroman;
4-(2-Aminoethyl)-7-benzyloxy-chroman;
4-(3-Aminopropyl)-7-benzyloxy-chroman;
4-[(Methylamino)methyl]-7-benzyloxy-chroman;
4-[2-(Methylamino)ethyl]-7-benzyloxy-chroman;
4-[3-(Methylamino)propyl]-7-benzyloxy-chroman;
4-Aminomethyl-7-(3-chlorobenzyloxy)-chroman;
4-(2-Aminoethyl)-7-(3-chlorobenzyloxy)-chroman;
4-[(Methylamino)methyl]-7-(3-chlorobenzyloxy)-chroman;
4-Aminomethyl-7-(3-fluorobenzyloxy)-chroman;
4-(2-Amino ethyl)-7-(3-fluorobenzyloxy)-chroman;
4-[(Methylamino)methyl]-7-(3-fluorobenzyloxy)-chroman;
4[2-(Methylamino)ethyl]-7-(3-fluorobenzyloxy)-chroman;
if the case, as a single optical isomer or a mixture thereof,
and the pharmaceutically acceptable salts thereof.

7. A compound of claim 6 selected from
4-[(Hydrazinocarbonyl)methyl]-7-benzyloxy-2H-chromen-2-one;
4-[(Aminocarbonyl)methyl]-7-(3-fluorobenzyloxy)-2H-chromen-2-one;
4-[(Dimethylaminocarbonyl)methyl]-7-(3-chlorobenzyloxy)-2H-chromen-2-one;
4-[(Dimethylaminocarbonyl)methyl]-7-benzyloxy-2H-chromene;
4-Aminomethyl-7-(3-chlorobenzyloxy)-2H-chromen-2-one;
4-[(Methylamino)methyl]-7-(3-fluorobenzyloxy)-2H-chromen-2-one;
4-(2-Aminoethyl)-7-(3-chlorobenzyloxy)-2H-chromen-2-one;
4-[(Methylamino)methyl]-7-(3-chlorobenzyloxy)-2H-chromen-2-one;
4-[2-(Methylamino)ethyl]-7-(3-chlorobenzyloxy)-2H-chromen-2-one,
if the case, as a single optical isomer or a mixture thereof,
and the pharmaceutically acceptable salts thereof.

8. A pharmaceutical composition containing, as an active principle, a compound of formula (I) as defined in claim 1 if the case as a single optical isomer or a mixture thereof, or a pharmaceutically acceptable salt thereof, in addition to a suitable carrier and/or diluent.

9. A composition of claim 8 wherein said composition contains one or more therapeutic agents in addition to the compound of formula (I).

10. A compound of formula (I), as defined in claim 1, if the case as a single optical isomer or a mixture thereof, or a pharmaceutically acceptable salt thereof, for use as an active therapeutic substance.

11. A compound of claim 1 for use in the manufacture of a medicament for the prevention and treatment of CNS degenerative disorders.

12. A compound of claim 1 for use as a medicament in Parkinson's disease, Alzheimer's disease, restless leg syndrome, epilepsy, amylotrophic lateral sclerosis, stroke, attention deficit hyperactivity disorders, drug addiction, smoking cessation or obesity.

13. A method for preventing CNS degenerative disorders comprising administering to a host in need thereof an effective dose of a compound of claim 1.

14. A method of claim 13 wherein the CNS degenerative disorders include Parkinson's disease, Alzheimer's disease, restless leg syndrome, epilepsy, amylotrophic lateral sclerosis, stroke, attention deficit hyperactivity disorders, drug addiction, smoking cessation and obesity.
